(19) United States
(12) Patent Application Publication
(10) Pub. No.: US 2008/0033007 A1
(43) Pub. Date: Feb. 7, 2008

(54) Substituted Tetrahydroisoquinoline Compounds for Cancer Therapy

(76) Inventors: Duane D. Miller, Germantown, TN (US); Eldon E. Geisert, Germantown, TN (US); Charles R. Yates, Collierville, TN (US); Renukadevi Patil, Memphis, TN (US); William E. Orr, Arlington, TN (US); XiangDi Wang, Germantown, TN (US); Fei Ma, Memphis, TN (US); Oleg Kirichenko, Littleton, CO (US)

Correspondence Address: DONNA J. RUSSELL, 1492 ANTHONY WAY, MT. JULIET, TN, 37122, US

(21) Appl. No.: 11/737,126

(22) Filed: Apr. 18, 2007

Related U.S. Application Data
(63) Continuation-in-part of application No. 10/389,651, filed on Mar. 13, 2003, now Pat. No. 7,241,774.
(60) Provisional application No. 60/363,952, filed on Mar. 13, 2002.
(60) Provisional application No. 60/745,008, filed on Apr. 18, 2006.

(51) Int. Cl. A61K 31/472; A61P 35/00; C07D 217/02; C07D 217/26; C07D 411/10

(52) U.S. Cl. 514/307; 546/148; 546/149

(57) ABSTRACT

Disclosed are compounds that are effective for selectively killing cancer cells. Compounds have been demonstrated to be especially effective for killing glioma cells, while exhibiting low toxicity to normal cells.

### CROSS REFERENCE TO RELATED APPLICATIONS

This application is a continuation-in-part of U.S. patent application Ser. No. 10/389,651, which claims the benefit of U.S. Provisional Patent Application Ser. No. 60/363,952, filed Mar. 13, 2002. This application also claims the benefit of priority of earlier-filed U.S. Provisional Patent Application No. 60/745,008, filed Apr. 18, 2006. U.S. patent application Ser. No. 10/389,651 corresponds to U.S. Publication No. 2004/0019078, which is incorporated herein by reference.

### FIELD OF THE INVENTION

The present invention relates generally to the synthesis of substituted tetrahydroisoquinoline compounds and to the use of substituted tetrahydro-isoquinoline compounds for the treatment of cancer.

### BACKGROUND OF THE INVENTION

Despite decades of research, the prognosis of most of the 17,500 patients diagnosed annually with brain cancer is very poor. The mortality rate of brain cancer patients is about 80 percent, second only to lung cancer patients whose mortality rate is about 85 percent (Bethune et al., Pharm. Res. 16(6):896-905 (1999)). The standard treatment is surgical excision and radiation therapy with or without adjuvant chemotherapy. Unfortunately, the Food and Drug Administration has not approved many new drugs for treatment of brain cancer over the last three decades. As of 1997, carmustine (“BCNU”), lomustine (“CCNU”), procarbazine, and vincristine were still the most commonly used drugs for both newly diagnosed and recurrent gliomas (Prados et al., Sem. Surgical Oncol. 14:88-95 (1998)). The use of adjuvant chemotherapy is currently controversial because very few patients respond to the standard chemotherapeutic protocols (Mason et al., J. Clin. Oncol. 15(12):3423-3427 (1997), although assays have been developed to identify which patients are likely to respond to chemotherapies. While these assays have improved survival rates slightly, the responsive tumors are in the minority and mortality rates remain high.

Numerous experimental trials have been attempted over the years with limited success. The most successful protocols (i.e., those that progressed to phase II clinical trials) involved combination therapies. The majority of single therapies did not pass phase I trials. Molecules such as the prototypical isoquinoline PX1195, benzodiazepine R₀₅₋₄₈₆₄, and the recently reported pyrrolobenzoxazepine NF 182 (Zisterer et al., Biochem. Pharmacol. 55:397-403 (1998)) form a class of agents that bind to the peripheral benzodiazepine receptors (“PBR”) and possess antiproliferative activity toward C6 glioma cells. The EC₅₀ values for these agents are 73 μM, 95 μM, and 37.5 μM, respectively. While these PBR ligands have been useful as brain cancer imaging agents (Olsen et al., Cancer Res. 48:5837-5841 (1988)), they have not been demonstrated to be useful for treating cancer. Thus, a need exists to identify compounds having improved antiproliferative/cytotoxic activity on brain and/or other forms of cancer.

What are still needed are agents for the effective treatment of cancer.

### SUMMARY OF THE INVENTION

One aspect of the present invention relates to a compound according to formula (I) as follows: 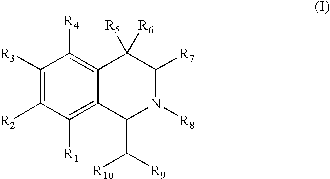
wherein:

R₁, R₂, R₃, and R₄ are independently hydrogen, hydroxyl, halide, alkyl, aryl, alkoxy, aryloxy, amino, alkylamino, or dialkylamino;

R₅ and R₆ are independently hydrogen, alkyl, or aryl;

R₇ is hydrogen, alkyl, alkylester, arylester, alkylamido, dialkylamido, arylamido, or R₆ and R₇ together are (—CH₂)ₖ— forming a ring structure fused with the N-hetero ring of (I), where k is either 3 or 4;

R₈ is hydrogen, alkyl, aryl, arylalkyl, 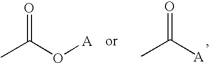
where A is substituted or unsubstituted alkyl, aryl, alkenyl, alkoxy, alkyloxy, or arylalkyl.

R₉ is 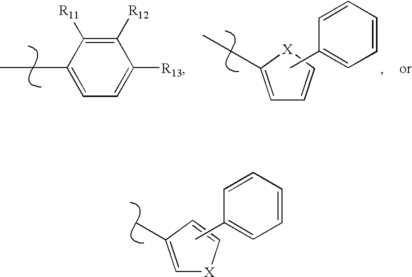
where X is oxygen, sulfur or nitrogen, 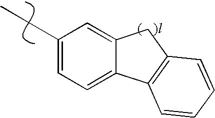
where l is 1 or 2, 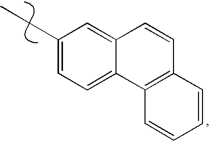
or R₈ and R₉ together are 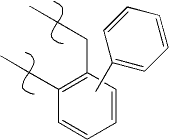
forming a ring structure fused with the N-hetero ring of (I):

R₁₀ is hydrogen or R₁ and R₁₀ together are (—CH₂)₂— forming a ring structure fused with both the benzene ring and the N-hetero ring of (I);

R₁₁, R₁₂, and R₁₃ are independently hydrogen, hydroxyl, halide, alkyl, arylalkyl, alkenyl, arylalkenyl, alkoxy, aryloxy, amino, alkylamino, dialkylamino, arylamino, aryl, cyclohexyl, or

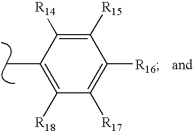

R₁₄, R₁₅, R₁₆, R₁₇, and R₁₈ are independently hydrogen, hydroxyl, halide, alkyl, alkoxy, amino, alkylamino, or dialkylamino.

A second aspect of the present invention relates to a compound according to formula (II) as follows: 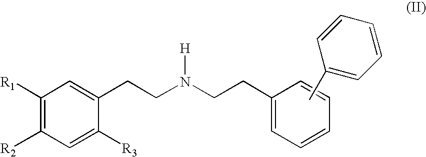
wherein R₁, R₂, and R₃ are independently hydrogen, hydroxyl, halide, alkyl, aryl, alkoxy, aryloxy, amino, alkylamino, or dialkylamino.

A third aspect of the present invention relates to a pharmaceutical composition that includes a pharmaceutically acceptable carrier and a compound of the present invention.

A fourth aspect of the present invention relates to a method of destroying a target cell that includes: providing a compound of the present invention and contacting a target cell with the compound under conditions effective to destroy the target cell.

A fifth aspect of the present invention relates to a method of treating or preventing a cancerous condition that includes: providing a compound of the present invention and administering an effective amount of the compound to a patient under conditions to treat an existing cancerous condition or prevent development of a cancerous condition.

A sixth aspect of the present invention relates to a method of preparing a tetrahydroisoquinoline compound of the present invention wherein R₉ is

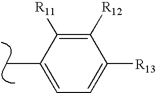

This method is carried out by reacting a precursor of formula (IA) 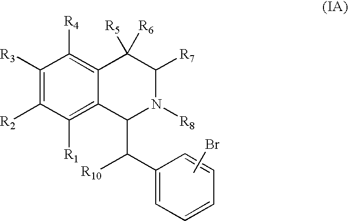
with Q-B(OH)₂ under conditions effective to replace the bromo group with -Q at the ortho, meta, or para position, wherein Q is alkyl, arylalkyl, alkenyl, arylalkenyl, alkoxy, aryloxy, aryl, cyclohexyl, or

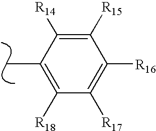

A seventh aspect of the present invention relates to another method of preparing a tetrahydroisoquinoline compound of the present invention. This method is carried out by treating a precursor of formula (IB) 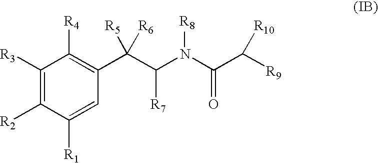
under conditions effective to form a six-membered N-hetero ring fused with the phenyl, thereby forming the compound of formula (I).

An eighth aspect of the present invention relates to a method of preparing a compound according to formula (II) of the present invention. This method is carried out by treating a compound having the structure 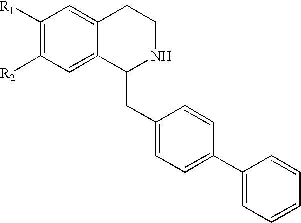
under conditions effective to open the N-hetero ring between C-1 and the phenyl ring, thereby forming the compound according to formula (II).

Compounds of the present invention have been demonstrated to be effective, both in vitro and in vivo, in destroying cancer cells and thus for the treatment of various forms of cancer including, without limitation, brain cancer, lung cancer, breast cancer, prostate cancer, cervical cancer. Several compounds of the present invention have shown greater efficacy than conventional therapeutics in destroying cancer cells and, significantly, causes less harm to normal cells. Without being bound by theory, it is believed that the compounds of the present invention are capable of destroying mitochondria and thereby disrupting cellular metabolism in cancer cells, eventually leading to ablation of the treated cells. As a result of their efficacy, the compounds of the present invention can afford effective treatments for various forms of cancer, such as gliomas or glioblastomas, which historically have low long-term survival rates.

### BRIEF DESCRIPTION OF THE DRAWINGS

FIGS. 1A-E illustrate the growth of C6 glioma in the brains of rats with vehicle only as control (1A-C), the anti-cancer drug BCNU (1D), and 6,7-bis-hydroxy-1-biphenyl-4-ylmethyl-1,2,3,4-tetrahydro-isoquinoline hydrochloride (designated MMGS-155) (1E). Approximately 5×10⁴ C6 glioma cells were transplanted into a rat brain via a cannula followed by treatment for seven days. Tumor cells are depicted by staining for beta galactosidase. Regions of the control treatment are shown at higher magnifications (1B and 1C) to illustrate the invasiveness of the glioma tumor. At the highest magnification (1C), the infiltration of the tumor along blood vessels can be observed.

FIG. 2 is a graph depicting the maximum extent of tumor in a single section. The control, BCNU, and 6,7-bis-hydroxy-1-biphenyl-4-ylmethyl-1,2-,3,4-tetrahydro-isoquinoline hydrochloride (MMGS-155) treatments are as described for FIG. 1. There was no significant size (area) difference in the control and BCNU-treated rats. The rats treated with MMGS-155 had tumors significantly smaller than the control groups (Mann-Whitney U test, p=0.009).

FIG. 3 is an enlargement of FIG. 1F, illustrating that the transplanted glioma are virtually absent. Of note, the brain tissue at the end of the cannula has substantially maintained its integrity despite the initial infiltration of the glioma cells and the subsequent drug administration.

FIGS. 4A-B illustrate the activity of 6,7-bis-hydroxy-1-biphenyl-4-ylmethyl-1,2,3,4-tetrahydro-isoquinoline hydrochloride (MMGS-155) on U87 glioma. At 18 hr after treatment with 3 μM 6,7-bis-hydroxy-1-biphen-yl-4-ylmethyl-1,2,3,4-tetrahydro-isoquinoline hydrochloride, the cells were fixed and stained with toluidine blue. Notable is the presence of vacuoles in the treated cells (4B) but not the control cells (4A).

FIGS. 5A-B illustrate the morphological change in U87 glioma cells after 6,7-bis-hydroxy-1-biphenyl-4-ylmethyl-1,2,3,4-tetrahydro-isoquinoline hydrochloride treatment. U87 glioma cells were treated with 5 μM 6,7-bis-hydroxy-1-biphenyl-4-ylmethyl-1,2,3,4-tetrahydroisoquinoline hydrochloride and at 18 hr the cells were stained with Hoechst dye. Many vacuoles were noted in the cytosol. This change can be seen as early as at 5 hr after treatment.

FIGS. 6A-B illustrate the morphological changes of glioma cells following 6,7-bis-hydroxy-1-biphenyl-4-ylmethyl-1,2,3,4-tetrahydroisoquinoline hydrochloride treatment. The images were taken by electron microscope. The vacuoles observed in FIGS. 4B and 5B can be identified here as ruptured mitochondria (arrows).

FIG. 7 is a graph illustrating the anti-proliferative activity of 6,7-bis-hydroxy-1-biphenyl-4-ylmethyl-1,2,3,4-tetrahydro-isoquinoline hydrochloride (MMGS-155) against cancer cell lines. The following cancer cell lines were treated with 6,7-bis-hydroxy-1-biphenyl-4-ylmethyl-1,2,3,-4-tetrahydro-isoquinoline hydrochloride for 96 hr at various concentrations: MCF7 (breast cancer, ●); A549 (lung epithelial cancer, ▪); HeLa (cervical cancer, ▴); LnCap (prostate cancer, ♦). Cell proliferation was measured and EC₅₀ values were estimated. 6,7-bis-hydroxy-1-biphenyl-4-ylmethy-1-1,2,3,4-tetrahydro-isoquinoline hydrochloride showed activity on all of the tested cancer cell lines.

FIG. 8 is a graph illustrating the anticancer activity of 6,7-bis-hydroxy-1-biphenyl-4-ylmethyl-1,2,3,4-tetrahydro-isoquinoline hydrochloride on several different human glioblastoma cell lines.

FIGS. 9A-B illustrate the comparision of anti-proliferative activity of 6,7-bis-hydroxy-1-biphenyl-4-ylmethyl-1,2,3,4-tetrahydro-isoquinoline hydrochloride (MMGS-155) and BCNU against C6 glioma cells. In FIG. 9A, C6 glioma cells were treated with MMGS-155 (▪)and BCNU (●) for 96 hr. 6,7-bis-hydroxy-1-biphenyl-4-ylmethyl-1,2,3,4-tetrahydroisoquinoline hydrochloride showed greater anti-proliferative activity than BCNU. In FIG. 9B, the selectivity of BCNU and 6,7-bis-hydroxy-1-biphenyl-4-ylmethyl-1,2,3,4-tetrahydro-isoquinoline hydrochloride on C6 glioma cells relative to primary cultured cortical astrocytes is shown for concentrations at EC₅₀.

FIG. 10 illustrates the animal toxicity test of 6,7-bis-hydroxy-1-biphenyl-4-ylmethyl-1,2,3,4-tetrahydro-isoquinoline hydrochloride. The drug was delivered directly into the striatum of rats at a concentration of 5 μM, two times greater than the estimated EC₅₀ value. After implanting the mini-osmotic pump with MMGS-155 for five days, rats did not show any sign of brain damage. The rats were sacrificed, brain sections were investigated for any major tissue change in the brain. Brains were fixed in 4% paraformaldehyde, cut in sections and stained by the Nissl method.

FIGS. 11-13 illustrate reaction steps used to synthesize compounds having anti-glioma activity.

FIG. 14 is a series of chemical structures for compounds tested for anti-glioma activity, the previously known PBR ligands PK11195 (9), R_{O}5-4864 (10), and NF 182 (11) being used as reference compounds against which the activity of the synthesized tetrahydroisoquinoline derivatives could be measured.

FIGS. 15A and 15B are dose-response curves. The dose response curve in FIG. 15A illustrates the selectivity of 1-(biphenyl-4-ylmethyl)-1,2,3,4-tetrahydroisoquinoline-6,7-diol for C6 glioma (▪) compared to neonatal astrocytes (●). The dose response curve in 15B illustrates the increased relative potency of 1-(biphenyl-4-ylmethyl)-1,2,3,4-tetrahydroisoquinoline-6,7-diol (▪) compared to the glioma chemotherapeutic agent carmustine or BCNU (●) against C6 glioma. Dose response curves were generated using Scientist® (MicroMath, Inc.) software, Version 2.01.

FIG. 16 is a dose-response curve illustrating that the antiglioma activity of C6 cells is also applicable to human gliomas. The human glioblastoma multiforme cell line T98G (American Type Culture Collection) was used. The dose-response curve and IC₅₀ value were attained by curve fitting the data to an inhibitory activity model in WinNonlin® (Pharsight Corporation). The curve drawn represents the best-fit curve derived from WinNonlin® and the error in the IC₅₀ value represents the 95% CI derived from this curve fit.

FIG. 17-FIG. 20 represent reaction schemes used to synthesize antiglioma compounds of the present invention.

### DETAILED DESCRIPTION OF THE INVENTION

The present invention relates generally to substituted tetrahydro-isoquinoline compounds, compositions that contain such compounds, methods for preparing such compounds, and their use for cancer therapy.

The compounds of the present invention include compounds of formulae (I) and (II) as set forth below. The compounds of formula (I) include: 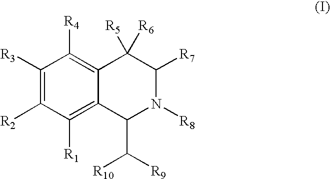
wherein,

R₁, R₂, R₃, and R₄ are independently hydrogen, hydroxyl, halide, alkyl, aryl, alkoxy, aryloxy, amino, alkylamino, or dialkylamino;

R₅ and R₆ are independently hydrogen, alkyl, or aryl;

R₇ is hydrogen, alkyl, alkylester, arylester, alkylamido, dialkylamido, arylamido, or R₆ and R₇ together are —(CH₂)ₖ— forming a ring structure fused with the N-hetero ring of (I), where k is either 3 or 4;

R₈ is hydrogen, alkyl, aryl, arylalkyl, 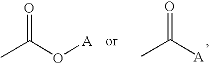
where A is substituted or unsubstituted alkyl, aryl, alkenyl, alkoxy, alkyloxy, or arylalkyl.

R₉ is 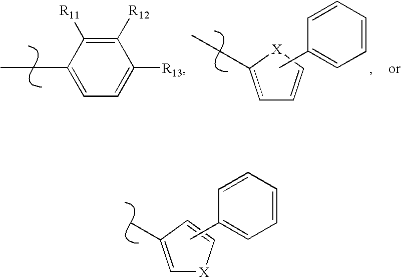
where X is oxygen, sulfur or nitrogen, 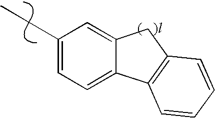
where l is 1 or 2, 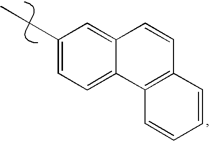
or R₈ and R₉ together are 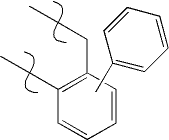
forming a ring structure fused with the N-hetero ring of (I):

R₁₀ is hydrogen or R₁ and R₁₀ together are (—CH₂)₂— forming a ring structure fused with both the benzene ring and the N-hetero ring of (I);

R₁₁, R₁₂, and R₁₃ are independently hydrogen, hydroxyl, halide, alkyl, arylalkyl, alkenyl, alkoxy, aryloxy, amino, alkylamino, dialkylamino, arylamino, aryl, cyclohexyl, or

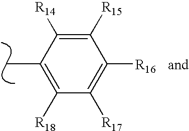

R₁₄, R₁₅, R₁₆, R₁₇, and R₁₈ are independently hydrogen, hydroxyl, halide, alkyl, alkoxy, amino, alkylamino, or dialkylamino.

The compounds of formula (II) include: 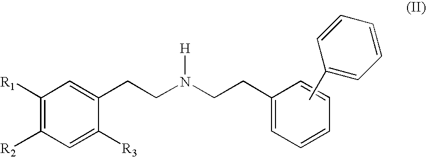
wherein R₁, R₂, and R₃ are independently hydrogen, hydroxyl, halide, alkyl, aryl, alkoxy, aryloxy, amino, alkylamino, or dialkylamino.

As used herein, the term “halide” unless otherwise specified, refers to a substituent that is an element from Group VIIA (e.g., fluorine, chlorine, bromine, iodine).

As used herein, the term “alkyl” unless otherwise specified refers to both straight chains alkyls that have the formula (—CH₂)ₓCH₃ where x is from 0 to 9, and branched chain alkyls that have the formula as defined above for straight chain alkyls, except that one or more CH₂ groups are replaced by CHW groups where W is an alkyl side chain. Exemplary alkyl groups include, without limitation, methyl, ethyl, propyl, 1-methylpropyl, 2-methylpropyl, butyl, t-butyl, pentyl, hexyl, heptyl, octyl, nonyl, decyl, etc. Alkyl groups that are substituents of a larger R-group, e.g., alkoxy, alkylester, alkylamino, alkylamido, etc. can be an alkyl group as defined above.

As used herein, the term “alkenyl”, unless otherwise specified, refers to both straight chain alkenyls that have the formula —(CH₂)ₓₐCH═CH(CH₂)_{xb}CH₃ where xa and xb each are from 0 to 7 and (xa+xb) is not more than about 7; and branched chain alkenyls have the formula as defined above for straight chain alkenyl, except that one or more CH₂ groups are replaced by CHW groups or a CH group is replaced by a CW group, where W is an alkyl side chain. Exemplary alkenyl groups include, without limitation, —CHCHC(CH₃)₃, and (—CHCH)ₙCH₃ where n is an integer from 1 to 4. Alkenyl groups also include those possessing multiple alkene double bonds, such as di-enes, tri-enes, etc. Alkenyl groups that are substituents of a larger R-group can be an alkenyl group as defined above.

As used herein, the term “aryl” refers to single, multiple, or fused ring structures containing one or more aromatic or heteroaromatic rings. Exemplary aryls include, without limitation, phenyls, indenes, pyrroles, imidazoles, oxazoles, pyrrazoles, pyridines, pyrimidines, pyrrolidines, piperidines, thiophenes, furans, napthals, bi-phenyls, tri-phenyls, and indoles. The aromatic or heteroaromatic rings can include mono-, di-, or tri-substitutions of the ring located at the ortho, meta, or para positions. Preferred aryls include, without limitation: 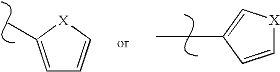
where X is oxygen, sulfur, or nitrogen, 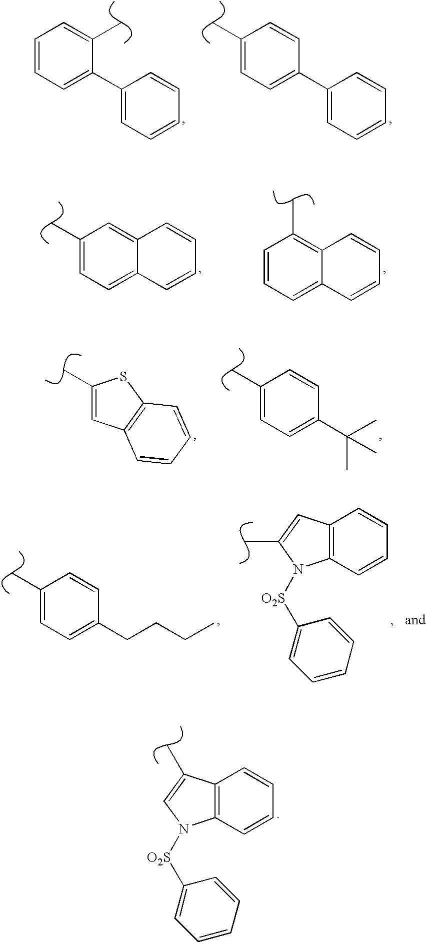
Aryl groups that are substituents of a larger R-group, e.g., aryloxy, arylamino, arylalkyl, arylamido, etc., can be an aryl group of the type defined above.

The compounds of the present invention can be prepared as either a racemic mixture, which includes both (+) and (−) stereoisomers, or as a substantially pure stereoisomer. By racemic mixture, it is intended that the mixture contain an approximately 1:1 ratio of the (+) and (−) isomers. By substantially pure, it is intended that one isomer is prepared such that it is at least about 85 percent pure, more preferably at least about 90 pure, most preferably at least about 95, 96, 97, 98, or 99 percent pure relative to its stereoisomer. Thus, as an example, a preparation that includes 85 or more percent by weight of a (+)-isomer and 15 percent or less percent by weight of the corresponding (−)-isomer is considered substantially pure for the (+)-isomer and substantially free of the (−)-isomer. Purification of stereoisomers one from another can be performed using conventional high performance liquid chromatography techniques designed to separate the stereoisomers or by using substantially pure starting materials or intermediates, thereby affording substantially pure final products.

When R₉ is 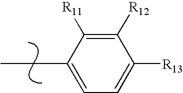
preferred compounds of the present invention are characterized by mono-substitution of the phenyl ring (of the R₉ substituent). Thus, when R₁₃ is a substituent other than hydrogen, R₁₁ and R₁₂ are both hydrogen; when R₁₂ is a substituent other than hydrogen, R₁₁ and R₁₃ are both hydrogen; and when R₁₁ is a substituent other than hydrogen, R₁₂ and R₁₃ are both hydrogen.

Preferred compounds of the present invention include the following: 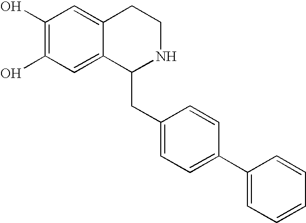
6,7-bis-hydroxy-1-biphenyl-4-ylmethyl-1,2,3,4-tetrahydro-isoquinoline; 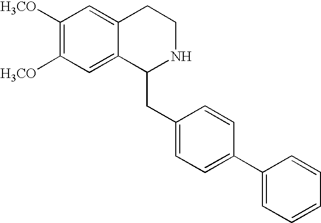
6,7-bis-methoxy-1-biphenyl-4-ylmethyl-1,2,3,4-tetrahydro-isoquinoline; 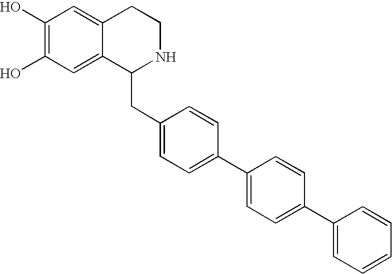
1-[1,1′;4′,141]terphenyl-4-ylmethyl-1,2,3,4-tetrahydro-isoquinolin-e-6,7-diol; 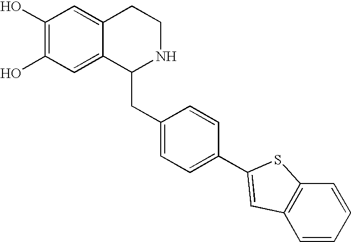
1-(4-benzo[b]thiophen-2-yl-benzyl)-1,2,3,4-tetrahydro-isoquinoline-6,7-diol; 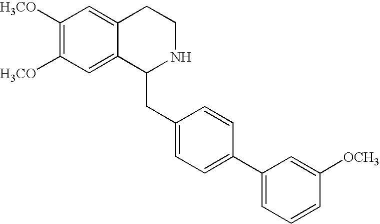
6,7-dimethoxy-1-(3′-methoxy-biphenyl-4-ylmethyl)-1,2,3,4-tetrahydro-isoquinoline; 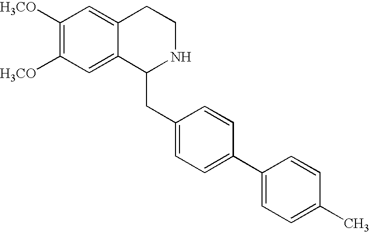
6,7-dimethoxy-1-(4′-methyl-biphenyl-4-ylmethyl)-1,2,3,4-tetrahydro-isoquinoline; 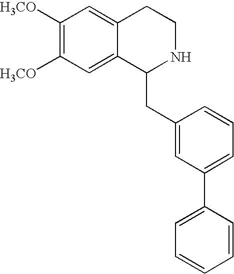
1-biphenyl-3-ylmethyl-6,7-dimethoxy-1,2,3,4-tetrahydro-isoquinoline; and 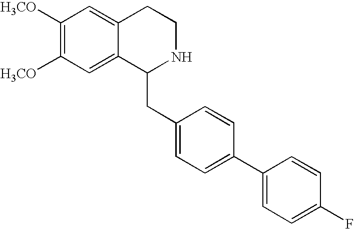
1-(4′-fluoro-biphenyl-4-ylmethyl)-6,7-dimethoxy-1,2,3,4-tetrahydro-isoquinoline.

Compounds of the present invention can be in the form of neutral compounds or in the form of salts. Pharmaceutically acceptable salts include those formed with free amino groups or with free carboxyl groups. For example, amino-salts may include, without limitation, hydrochloric, hydrobromic, phosphoric, acetic, oxalic, tartaric acids, etc. Carboxyl-salts may include, for example, sodium, potassium, ammonium, calcium, ferric hydroxides, isopropylamine, triethylamine, 2-ethylamino ethanol, histidine, procaine, etc. Because the structure of formulae (I) and (II) may include an available nitrogen (i.e., in the N-hetero ring of formula (I) or the secondary amino group of formula (II)), amino-salts may be preferred. Suitable salts can be prepared in accordance with known procedures.

Generally, the compounds of the present invention can be synthesized according to the procedures shown in the synthesis schemes below and other reaction schemes known to those of skill in the art for effecting a substitution of one group for another on an intermediate compound.

Compounds of the present invention where R₉ is phenyl, R₁₁ and R₁₂ are hydrogen, and R₁₃ is alkyl, arylalkyl, alkenyl, alkoxy, aryloxy, amino, alkylamino, dialkylamino, arylamino, aryl, cyclohexyl, or 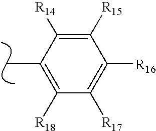
as described above, can be prepared according to Scheme I below.

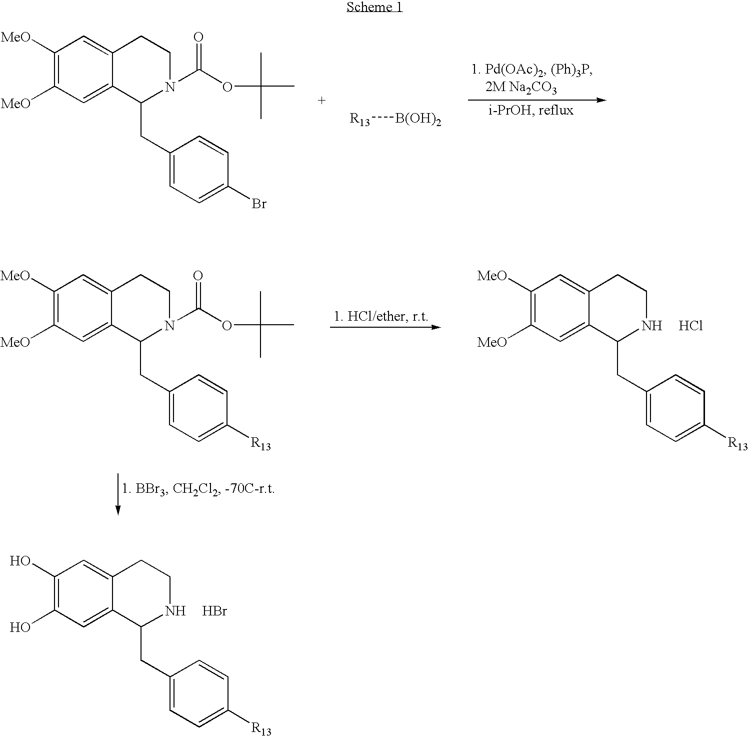

To prepare compounds of the present invention where R₉ is phenyl and either R₁₁ or R.₁₂ is alkyl, arylalkyl, alkenyl, alkoxy, aryloxy, amino, alkylamino, dialkylamino, arylamino, aryl, cyclohexyl, or 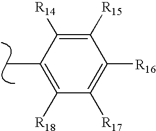
as described above, an analog of the starting compound shown in Scheme 1 can be used where the Br group (to be displaced) is bound to the phenyl group of R₉ at either the R₁₁ (ortho) position or the R₁₂ (meta) position, respectively (see Example 12 below).

Compounds of the present invention where R₉ is phenyl and R₁₃ is alkyl, arylalkyl, alkenyl, alkoxy, aryloxy, amino, alkylamino, dialkylamino, arylamino, aryl, cyclohexyl, or 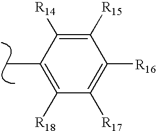
as described above, and where R₆and R₇ together are —(CH₂)₄— forming a ring structure fused with the N-hetero ring of (I), can be prepared according to Scheme 2 below.

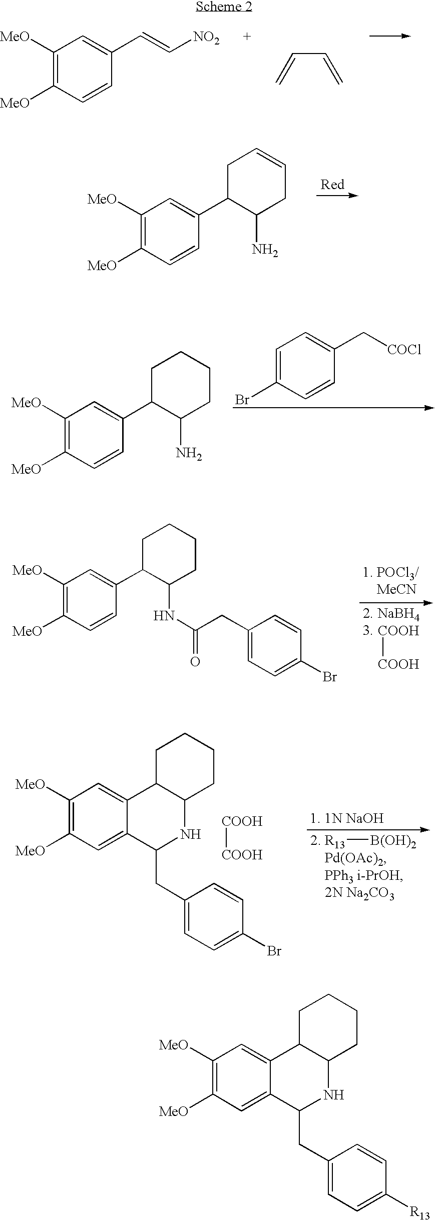

Compounds of the present invention where R₉ is 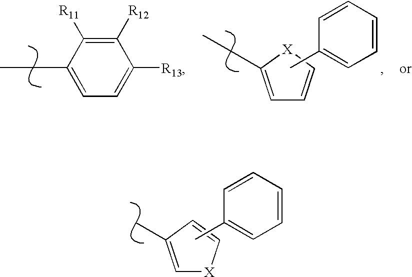
where X is oxygen, sulfur or nitrogen, 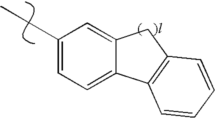
where l is 1 or 2, or 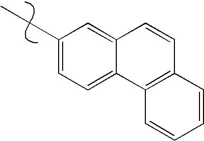
as described above, can be synthesized according to Scheme 3 below.

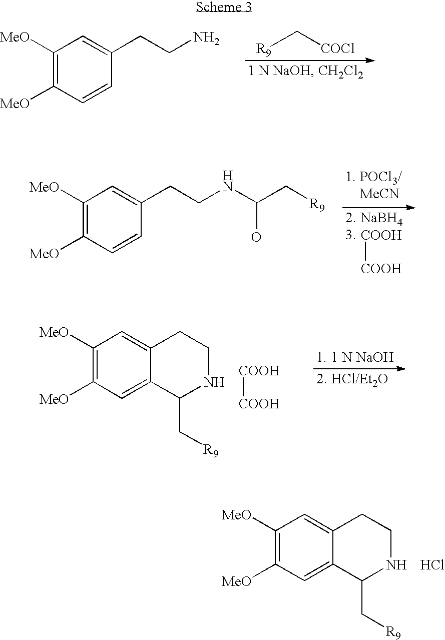

Compounds of the present invention where R₁ and R₁₀ together are —(CH₂)₂— forming a ring structure fused with both the benzene ring and the N-hetero ring of (I), can be prepared as shown in Scheme 4 below.

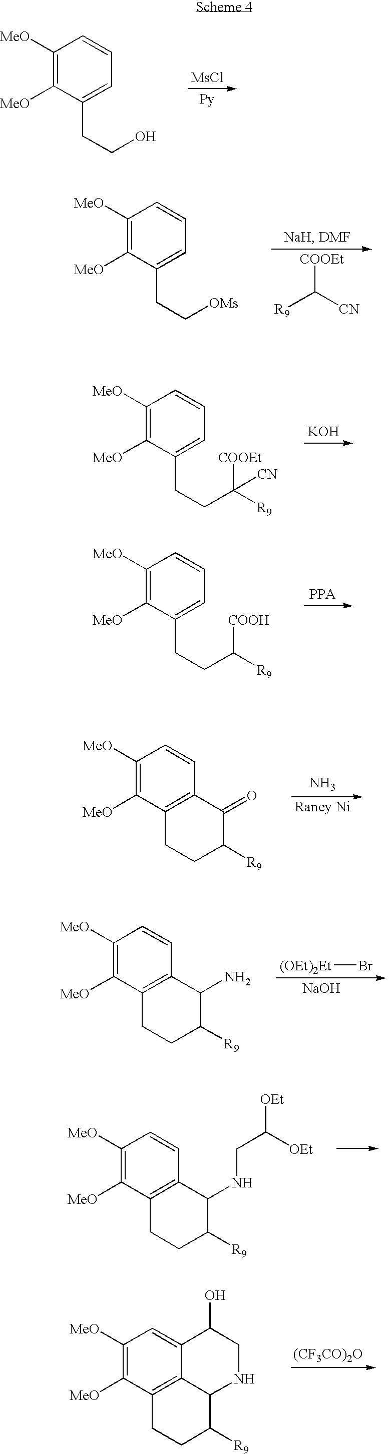

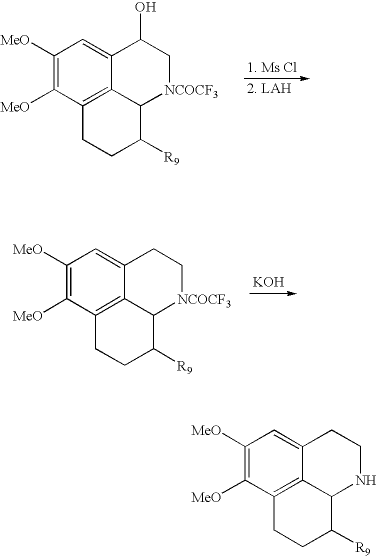

To prepare compounds of the present invention having R₁-R₇ groups as described above, starting reactants possessing various combinations of the recited R₁-R₇ substituents can be utilized. Compounds of formula (II) can be synthesized according to Scheme 5 below, beginning with a compound of formula (I) as shown in Scheme 1, with R₁₃ being a phenyl group.

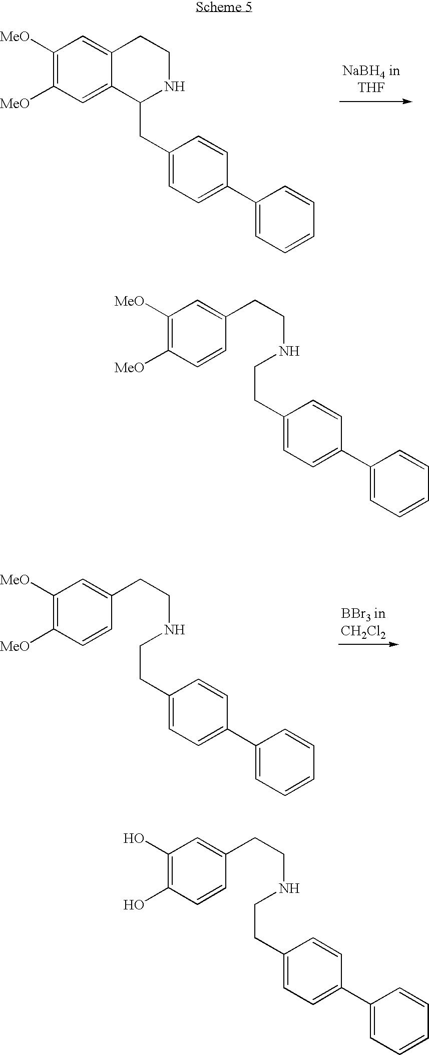

Compounds of the present invention may be formulated into or with a pharmaceutically acceptable carrier to afford a pharmaceutical composition of the present invention. The pharmaceutical composition may also include suitable excipients or stabilizers, and may be provided in solid or liquid form such as, tablets, capsules, powders, solutions, suspensions, or emulsions. Typically, such a composition will contain from about 0.01 to 99 percent by weight, preferably from about 10 to 75 percent by weight of more compounds as provided herein, together with one or more carriers and inert ingredients.

Solid unit dosage forms may be of a variety of conventional types known to those of skill in the pharmaceutical sciences. The solid form can be a capsule, such as an ordinary gelatin type containing the compounds of the present invention and a carrier. For example, lubricants and inert fillers such as, lactose, sucrose, or cornstarch may be used. In another embodiment, these compounds are formed into tablets with conventional tablet bases such as lactose, sucrose, or cornstarch in combination with binders like acacia, cornstarch, or gelatin, disintegrating agents, such as cornstarch, potato starch, or alginic acid, and a lubricant, such as, for example, stearic acid or magnesium stearate.

The compounds of the present invention may also be administered in injectable dosages by solution or suspension of these materials in a physiologically acceptable diluent with the pharmaceutical carrier. Such carriers may include sterile liquids, such as water and oils, with or without the addition of surfactant(s), adjuvant(s), excipients(s), or stabilizer(s). Illustrative oils are those of petroleum, animal, vegetable, or synthetic origin. For example, peanut oil, soybean oil, or mineral oil may be used. In general, water, saline, aqueous dextrose and related sugar solution, and glycols, such as propylene glycol or polyethylene glycol, are preferred liquid carriers, particularly for injectable solutions.

For use as aerosols, the compounds of the present invention in solution or suspension may be packaged in a pressurized aerosol container together with suitable propellants. For example, hydrocarbon propellants such as propane, butane, or isobutane may be used. The materials of the present invention also may be administered in a non-pressurized form such as in a nebulizer or atomizer.

Depending upon the treatment desired, compounds of the present invention can be administered orally, topically, transdermally, parenterally, subcutaneously, intravenously, intramuscularly, intra-peritoneally, by intranasal instillation, by intra-cavitary or intra-vesical instillation, by inhalation, intra-vaginally, intracranially (e.g., by cannula), intra-ocularly, intra-arterially, intra-lesionally, or by application to mucous membranes, such as, that of the nose, throat, and bronchial tubes. Other routes of administration may also be identified by those of ordinary skill in the art.

Compounds of the present invention are particularly useful in the treatment or prevention of various forms of cancer, including without limitation, brain cancer, lung cancer, breast cancer, prostate cancer, and cervical cancer. Compounds of the present invention have demonstrated the effect of selectively killing cancer cells at concentrations that cause little or no detectable harm to normal cells.

Thus, a further aspect of the present invention relates to a method of treating cancer that comprises administering to a human or animal patient a therapeutically effective amount of one or more compositions of the present invention to shrink or destroy a patient's tumor or tumors, destroy blood- or lymph-related cancer cells, or shrink or destroy one or more precancerous growths.

Compounds of the invention may be administered systemically or, alternatively, they can be administered directly to a specific site where cancer cells or precancerous cells are present. Thus, administering can be accomplished in any manner effective for delivering the compounds or the pharmaceutical compositions to the cancer cells or precancerous cells. When the compounds or pharmaceutical compositions of the present invention are administered to treat or prevent a cancerous condition, the pharmaceutical composition can also contain, or can be administered in conjunction with, other therapeutic agents or treatment regimen presently known or hereafter developed for the treatment of various types of cancer. Examples of other therapeutic agents or treatment regimen include, without limitation, radiation therapy, chemotherapy, surgical intervention, and combinations thereof.

Compositions within the scope of this invention include all compositions wherein the compound of the present invention is contained in an amount effective to achieve its intended purpose. While individual needs may vary, determination of optimal ranges of effective amounts of each component is within the skill of the art. Typical dosages comprise about 0.01 to about 100 mg/kg body wt. The preferred dosages comprise about 0.1 to about 100 mg/kg body wt. The most preferred dosages comprise about 1 to about 100 mg/kg body wt. Given the disclosure herein, it is well within the skill of those in the pharmaceutical and medical arts to determine a treatment regimen suitable for a particular human or animal patient.

The invention may be further described by means of the following non-limiting examples.

### EXAMPLE 1

### Preparation of the Racemic 6,7-bis-hydroxy-1-biphenyl-4-ylmethyl-1,2,3,4-t-etrahydro-isoquinoline Hydrochloride

The intermediate 2-biphenyl-4-yl-N-[2-(3,4-bis-benzyloxy-phenyl)-et-hyl]-acetamide (1) was prepared as follows: To a stirred solution of 2.85 g (7.7 mmol) of 3,4-dibenzyloxyphenethyl amine and 1.49 g (7 mmol) of 4-biphenylacetic acid in 20 ml of anhydrous dimethylformamide at 0° C., 2.5 ml (17.7 mmoles) of triethyl amine was added drop-wise and then 1.4 ml (7.7 mmol) of diethyl cyanophosphonate (90% purity) was added drop-wise. The reaction mixture was stirred for 22 hours and allowed to reach room temperature, and then it was poured into 300 ml of water. The precipitated solid was separated on a glass filter funnel, washed with water (3.times.70 ml), and air-dried overnight. This solid was recrystallized using hexanes/ethanol mixture to provide 3.1 g (84%) of grayish crystals, mp 142-144° C.

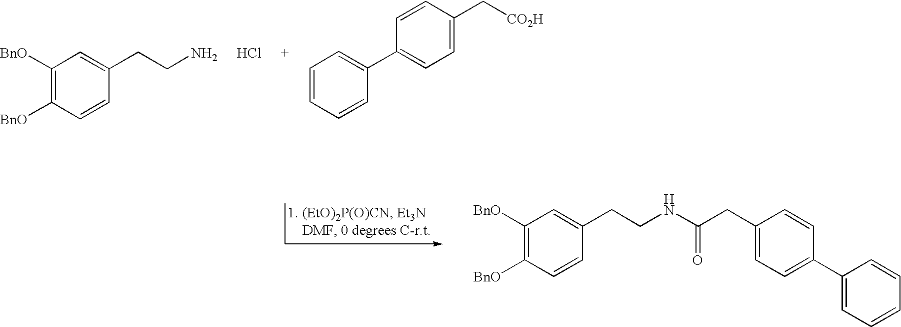

6,7-bis-benzyloxy-1-biphenyl-4-ylmethyl-1,2,3,4-tetrahydro-isoquino-line hydrochloride (2) was prepared as follows: A stirred solution of 3 g (5.69 mmol) of 2-biphenyl-4-yl-N-[2-(3,4-bis-benzyloxy-phenyl)-ethyl]-acetamide and 5.6 ml (60 mmol) of phosphorus oxychloride in 25 ml of anhydrous acetonitrile was refluxed for 20 hours. The reaction solution was evaporated under reduced pressure and the residue was dissolved in 25 ml of methanol. To this stirred solution at 0° C., 2.27 g (60 mmol) of sodium borohydride was added portionwise. The stirred reaction mixture was allowed to reach room temperature overnight and then it was poured in 150 ml of 10% aqueous HCl solution. The precipitate was collected on a glass filter funnel, washed with water (3×50 ml), and air-dried overnight. The solid was recrystallized using ether/methanol mixture to provide 2.56 g (82%) of off-white crystals, mp 187-189° C.

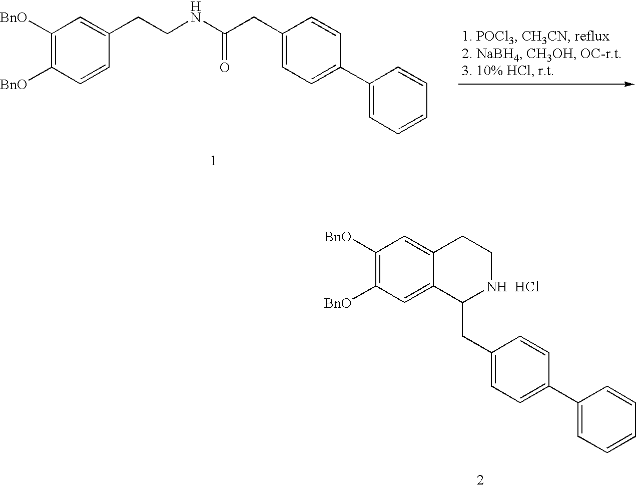

6,7-bis-hydroxy-1-biphenyl-4-ylmethyl-1,2,3,4-tetrahydro-isoquino-line hydrochloride (3) was prepared as follows: A stirred mixture of 2.5 g (4.56 mmol) of 6,7-bis-benzyloxy-1-biphenyl-4-ylmethyl-1,2,3,4-tetrahydroisoquinoline hydrochloride in 10 ml of concentrated aqueous HCl solution/10 ml of methanol was refluxed for 10 hours. The solvents were evaporated under reduced pressure, the solid residue was mixed with 10 ml of ether and separated on a glass filter funnel, and then washed with ether (2×10 ml). This solid was recrystallized using ether/methanol mixture to give 1.3 g (78%) of off-white crystals, mp 208-210° C.

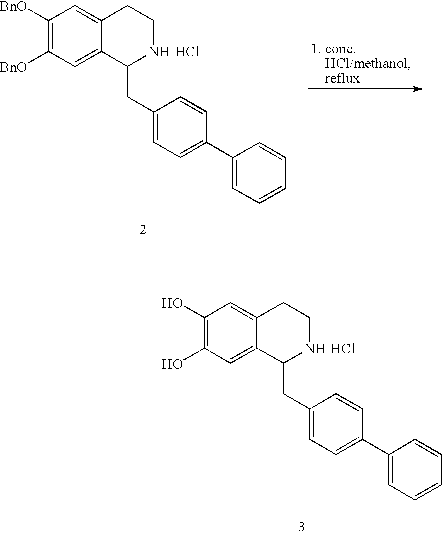

### EXAMPLE 2

### Chiral Separation of 6,7-bis-hydroxy-1-biphenyl-4-ylmethyl-1,2,3,4-tetrahydro-isoquinoline Hydrochloride

The chiral separation of the racemic mixture prepared in Example 1 was done on an HP 1100 HPLC system using a reverse-phase ChromTech Chiral-AGP column (150×4 mm). The column was operated in isocratic mode at a rate of 0.9 mL/min using a mobile phase of 7% acetonitrile in 10 mM sodium phosphate buffer, pH 5.5.

### EXAMPLE 3

### In Vivo Testing of 6,7-bis-hydroxy-1-biphenyl-4-ylmethyl-1,2,3,4-tetrahydro-isoquinoline Hydrochloride

A rat model system was developed to examine the in vivo efficacy of the compounds of the present invention.

The first step was to produce a cell line with a marker gene. A rat C6 glioma cell line was selected and stably transfected with a β-galactosidase construct. These cells were cultured and prepared for injection into the brain of adult Sprague-Dawley rats to simulate in vivo tumor development.

The animals were anesthetized and a cannula was placed into the brain. Approximately 5×10⁴ glioma cells were injected through the cannula. The cannula was then attached to an osmotic mini pump containing one of three treatment solutions: Hanks Balanced Salts (HBSS), HBSS plus 10 μM BCNU or HBSS with 7 μM 6,7-bis-hydroxy-1-biphenyl-4-ylmethyl-1,2,3,4-tetrahydro-isoquinoline hydrochloride. The mini osmotic pump delivered 1 μl/hr for 7 days. With the tubing used to connect the pump, there was approximately a 10 hour delay for the treatment to reach the brain.

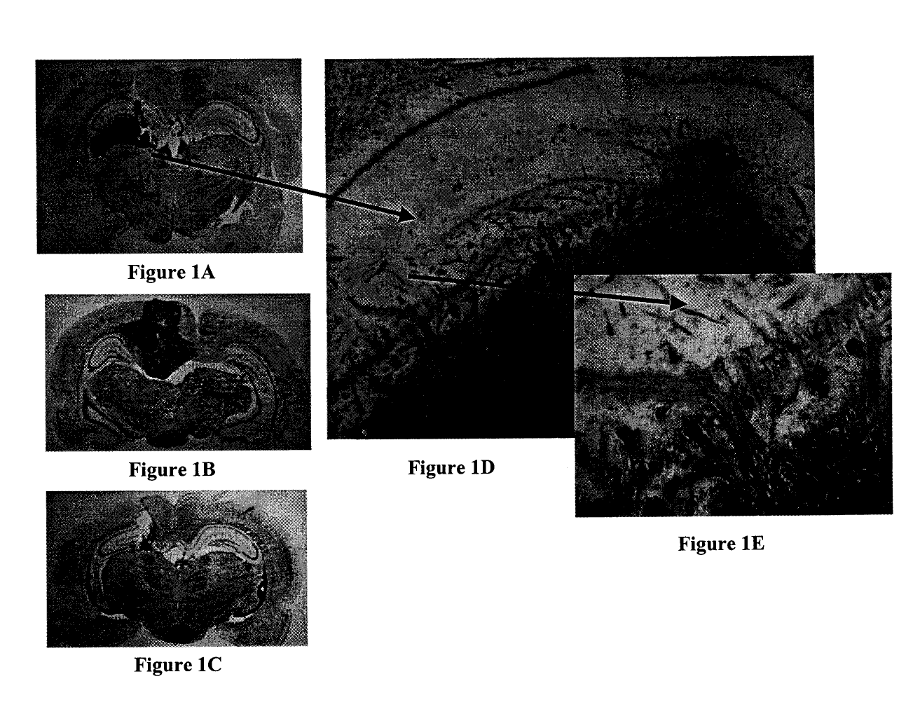

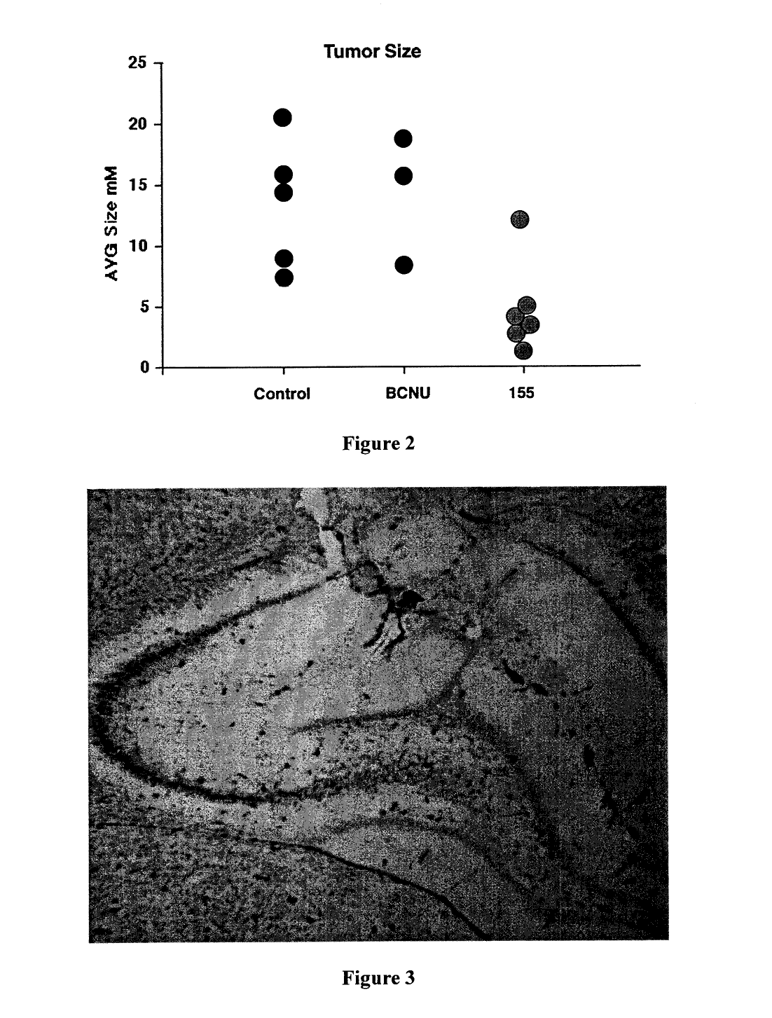

As can be seen in FIGS. 1A-C and 1D, respectively, the presence of the glioma is quite noticeable within the control and BCNU-treated brains. In addition to labeling the glioma, the beta galactosidase reaction also labels the choroid plexus in the lateral ventricles (the dark blue vertical stripes in the 6,7-bis-hydroxy-1-biphenyl-4-ylmethyl-1,2,3,4-tetrahydro-isoquinoline hydrochloride treated brain). As can be seen, the tumor is well established in the control brain and the BCNU-treated brain by 7 days. In the 6,7-bis-hydroxy-1-biphenyl-4-ylmethy-1-1,2,3,4-tetrahydro-isoquinoline hydrochloride treated brain, there is very little tumor present. The section with the largest extent of tumor visible in all of the cases was identified and the cross-sectional area of the tumor was measured. These results are displayed in FIG. 2. It is worth noting that these are cross-sectional areas and if volume were measured the differences between animals would be even greater. In some of the control brains the tumors had expanded to occupy nearly ¼ of the brain mass. The morphology of the hippocampus beneath the treatment cannula is shown in FIG. 3. Limited pathology is noted with the 6,7-bis-hydroxy-1-biphenyl-4-ylmethyl-1,2,3,4-tetrahydro-isoquinoline hydrochloride treatment. This compound causes a significant reduction in brain tumor size without killing the normal brain tissue.

### EXAMPLE 4

### In Vitro Analysis of 6,7-bis-hydroxy-1-biphenyl-4-ylmethyl-1,2,3,4-tetrahydro-isoquinoline Hydrochloride on Cancer Cell Lines

Below are described a series of experiments that point to a potential mechanism concerning the mechanism of action of 6,7-bis-hydroxy-1-biphenyl-4-ylmethyl-1, 2,3,4-tetrahydro-isoquinoline hydrochloride.

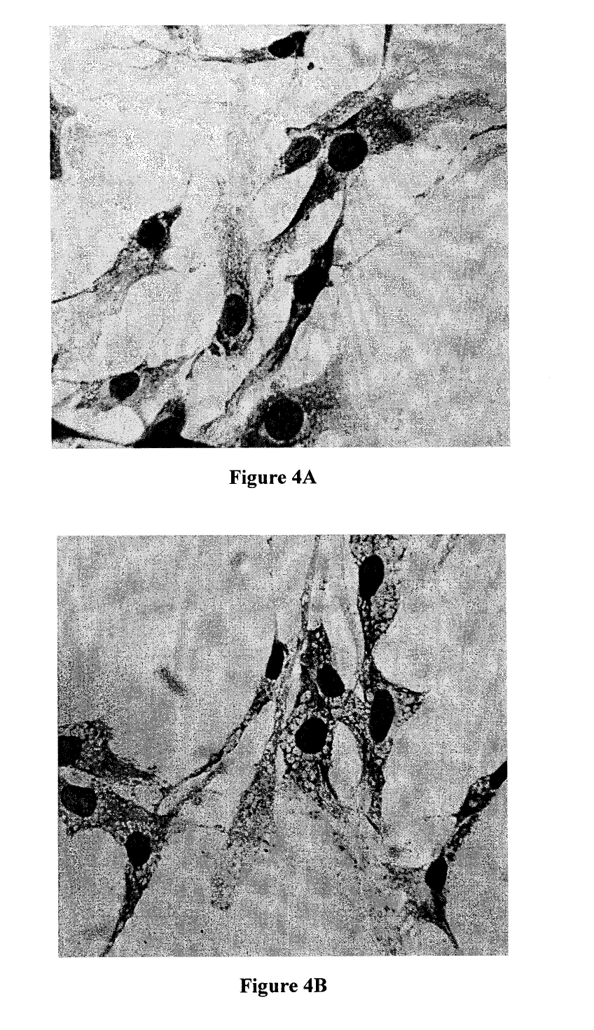

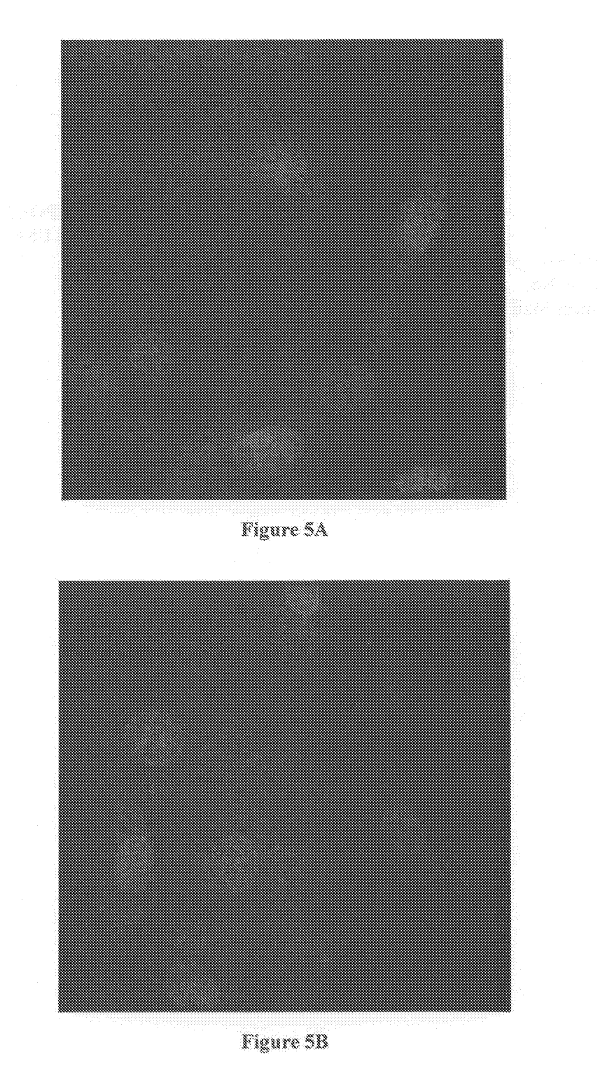

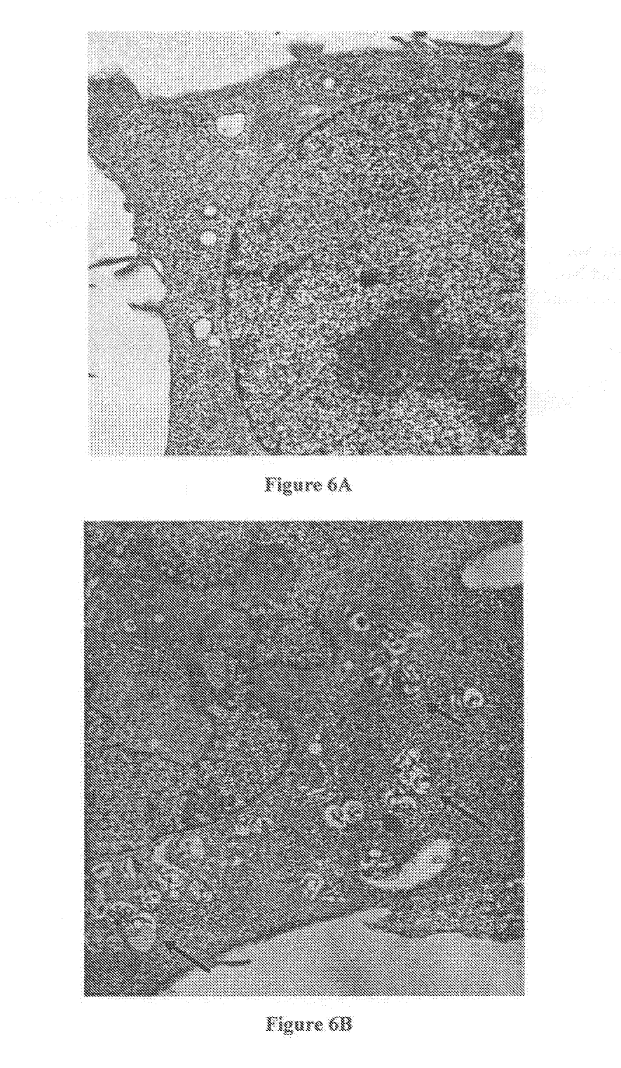

When human glioma are treated with 6,7-bis-hydroxy-1-biphenyl-4-ylmethyl-1,2,3,4-tetrahydro-isoquinoline hydrochloride in culture, they develop vacuoles (FIGS. 4A-B). These vacuoles do not contain significant amounts of DNS (FIGS. 5A-B). When the treated cells were examined at the electron microscopic level (FIGS. 6A-B), these vacuoles appear to contain fragments of mitochondria. The cells treated with 6,7-bis-hydroxy-1-biphenyl-4-ylmethyl-1,2,3,4-tetrahydro-isoquinoline hydrochloride were closely examined and the striking feature of the vacuoles contrasted by the absence of visible mitochondria. Thus, 6,7-bis-hydroxy-1-biphenyl-4-ylmethyl-1,2,3,4-tetrahydro-isoquinoline hydrochloride appears to cause mitochondrial disruption in the tumor cells, leading to tumor cell death.

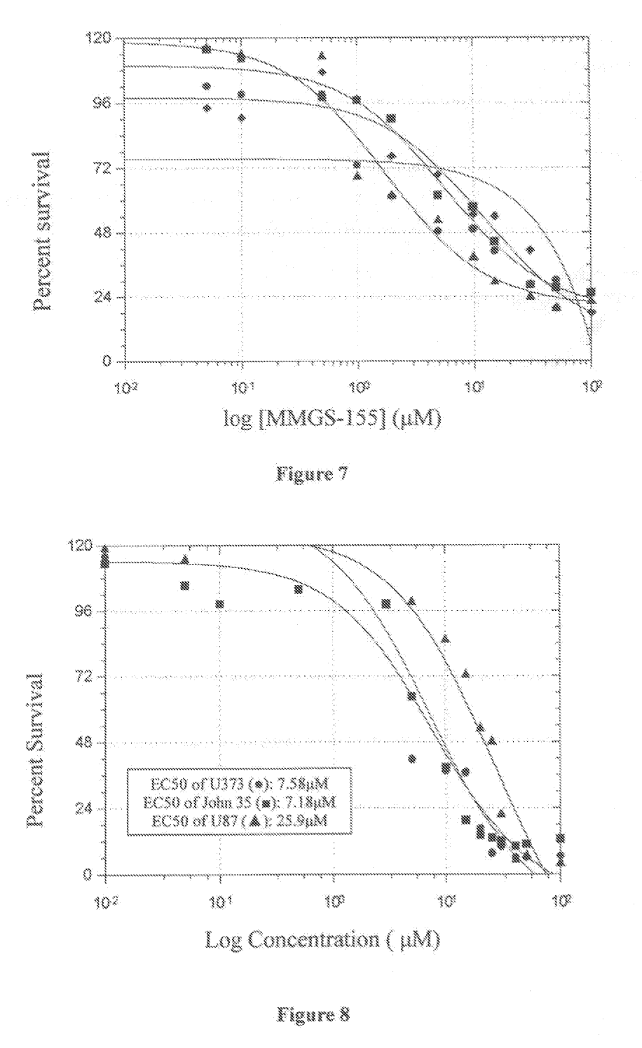

Given the above results, 6,7-bis-hydroxy-1-biphenyl-4-ylmethyl-1,2,-3,4-tetrahydro-isoquinoline hydrochloride was tested on a number of different types cancer cell lines as shown in FIG. 7. In every type of cancer tested by the inventors to date, including brain cancer (glioma and glioblastoma), breast cancer, lung epithelial cancer, cervical cancer, and prostate cancer cell lines, 6,7-bis-hydroxy-1-biphenyl-4-ylmethyl-1,2,3,4-tetrahydro-isoquinoline hydrochloride has killed the cancer cells in culture with an LD₅₀ of approximately 3 μM. Of special note is the fact that 6,7-bis-hydroxy-1-biphenyl-4-ylmethyl-1,2,3,4-tetrahydro-isoquinoline hydrochloride was effective in killing glioma cells (FIG. 8).

### EXAMPLE 5

### Differential Effect of 6,7-bis-hydroxy-1-biphenyl-4-ylmethyl-1,2,3,4-tetra-hydro-isoquinoline Hydrochloride on C6 Glioma Cells and Normal Brain Cells

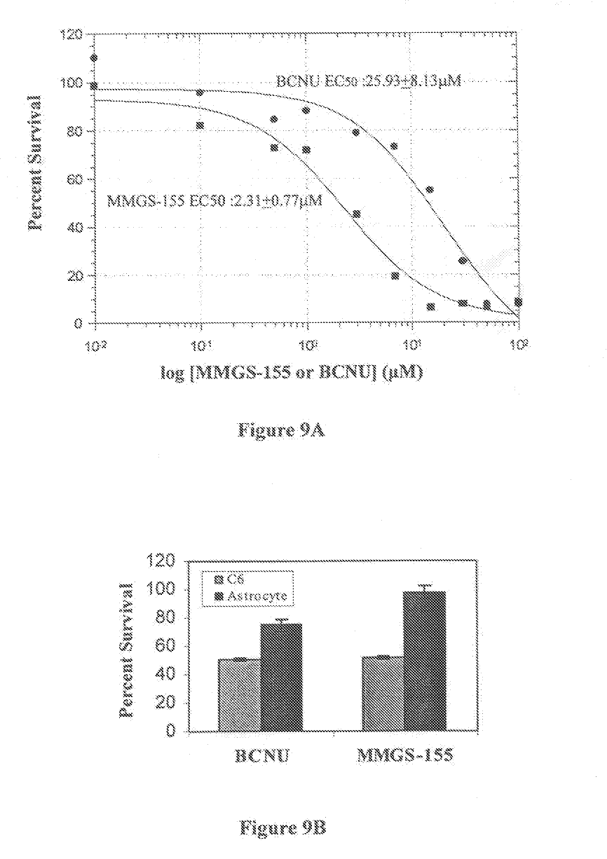

To further assess the differential effect of 6,7-bis-hydroxy-1-biphenyl-4-ylmethyl-1,2,3,4-tetrahydro-isoquinoline hydrochloride on cancer cells and normal cells, in vitro analysis was performed using 6,7-bis-hydroxy-1-biphenyl-4-ylmethyl-1,2,3,4-tetrahydro-isoquinoline hydrochloride and BCNU. C6 glioma cells were treated with varying doses of 6,7-bis-hydroxy-1-biphenyl-4-ylmethyl-1,2,3,4-tetrahydro-isoquinoline hydrochloride and BCNU for 96 hr and the survival rate of the C6 glioma cells was determined. As shown in FIG. 9A, 6,7-bis-hydroxy-1-biphenyl-4-ylmethyl-1,2,3,4-tetrahydro-isoquinoline hydrochloride possesses an EC₅₀ of 2.3±0.77 μM whereas BCNU possesses an EC₅₀ of 25.93±8.13 μM. Thus, confirming earlier results, 6,7-bis-hydroxy-1-biphenyl-4-ylmethyl-1,2,3,4-tetrahydro-isoquinoline hydrochloride is an order of magnitude more potent than BCNU.

Even more compelling, however, is the greater specificity that 6,7-bis-hydroxy-1-biphenyl-4-ylmethyl-1,2,3,4-tetrahydro-isoquinoline hydrochloride has for the C6 cells over primary cultured cortical astrocytes. As shown in FIG. 9B, at EC₅₀ concentrations for the C6 gliomas, 6,7-bis-hydroxy-1-biphenyl-4-ylmethyl-1,2,3,4-tetrahydro-isoquinoline hydrochloride caused only minimal destruction to astrocytes whereas BCNU caused significant destruction to astrocytes. The EC₅₀ concentration of 6,7-bis-hydroxy-1-biphenyl-4-ylmethyl-1,2,3,4-tetrahydro-isoquinoline hydrochloride for astrocytes was an order of magnitude higher (EC₅₀=27.29±5.29 μM). Thus, 6,7-bis-hydroxy-1-biphenyl-4-ylmethyl-1,2,3,4-tetrahydro-isoquinoline hydrochloride is a cytotoxic agent that not only has a broad range of activity, as demonstrated in Example 6, but also selectively targets cancer cells over normal cells.

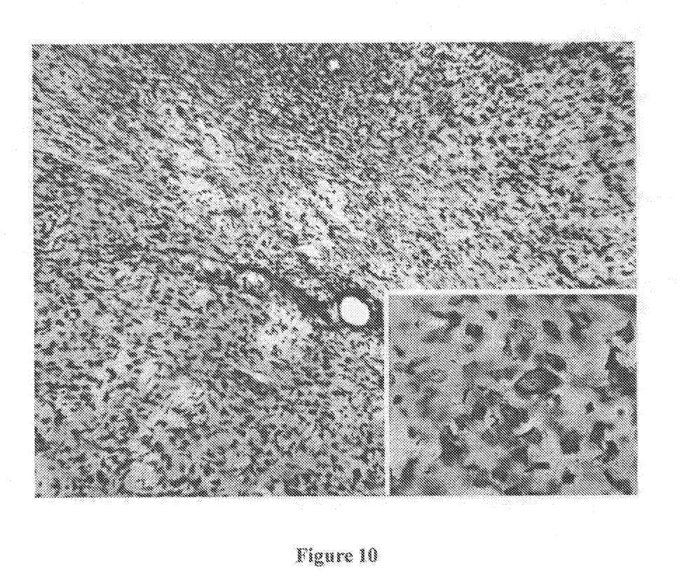

To further investigate these results, the in vivo effect of 6,7-bis-hydroxy-1-biphenyl-4-ylmethyl-1,2,3,4-tetrahydro-isoquinoline hydrochloride on healthy brain tissue was also measured. The drug was administered into the striatum of rats at 5 μM, which is approximately two times greater than the estimated EC₅) value. After implanting the mini-osmotic pump with MMGS-155 for five days, rats did not show any sign of brain damage. The rats were sacrificed and brain sections were investigated for any major tissue change in the brain. Brains were fixed in 4% paraformaldehyde, cut in sections, and stained by the Nissl method. As shown in FIG. 10, the brain tissue appears normal.

### EXAMPLE 6

### Preparation of the Racemic 1-[1,1′;4′,1″]Terphenyl-4-ylmethyl-1,2,3,4-tetrahydro-isoquinoline-6,7-diol Hydrobromide

The intermediate 1-(4-Bromo-benzyl)-6,7-dimethoxy-3,4-dihydro-1H-isoquinoline-2-carboxylic acid tert-butyl ester (11) was prepared as follows: To a stirred mixture of 2.76 g (6.92 mmoles) of 1-(p-bromobenzyl)-6,7,-dimethoxy-1,2,3,4-tetrahydro-isoquinoline hydrochloride and 1.81 g (1.2 mol. equiv.) of di-tert-butyldicarbonate in 100 ml of anhydrous tetrahydrofuran at room temperature, 2.3 ml (2.4 mol. equiv.) of triethylamine was added. The reaction mixture was stirred at room temperature for 12 hours and then the solvent was evaporated under reduced pressure. The residue was partitioned between 150 ml of ethyl acetate and 100 ml of 5% citric acid aqueous solution. After shaking, the organic layer was washed with 100 ml of water and 100 ml of brine, and then dried (Na₂SO₄). The solvent was evaporated under reduced pressure and the remaining oil was crystallized by trituration with n-hexanes. The solid was recrystallized using n-hexanes\ethyl acetate mixture to furnish 2.59 g (81%) of off-white solid, mp 93-95° C.

6,7-Dimethoxy-1-[1,1′;4′1″]terphenyl-4-ylmethyl-3,4-dihydro-1H-isoq-uinoline-2-carboxylic acid tert-butyl ester (12) was prepared as follows: A mixture of 0.462 g (1 mmol) of 1-(4-Bromo-benzyl)-6,7-dimethoxy-3,4-dihydro-1H-isoquinoline-2-carboxylic acid tert-butyl ester and 0.297 g (1.5 mol. equiv.) of 4-biphenylboronic acid in 4 ml of isopropanol was stirred under argon at room temperature for 30 min. To this mixture, 1 mg (0.45 mol %) of Palladium(II)acetate, 4 mg (1.3 mol %) of triphenylphosphine, and 0.6 ml (1.2 mol. equiv.) of 2M sodium bicarbonate aqueous solution were added successively and the mixture was refluxed with stirring for 6 hours. The solvent was evaporated under reduced pressure and the residue was partitioned between 50 ml of ethyl acetate and 25 ml of 5% sodium hydroxide aqueous solution. After shaking, the organic layer was washed with water (25 ml) and brine (25 ml), and then dried with sodium sulfate. The solvent was evaporated under reduced pressure and the remaining oil was crystallized by trituration with n-hexanes. The solid was recrystallized using ethyl acetate\n-hexanes mixture to give 0.40 g (75%) of white solid, mp 153-155° C.

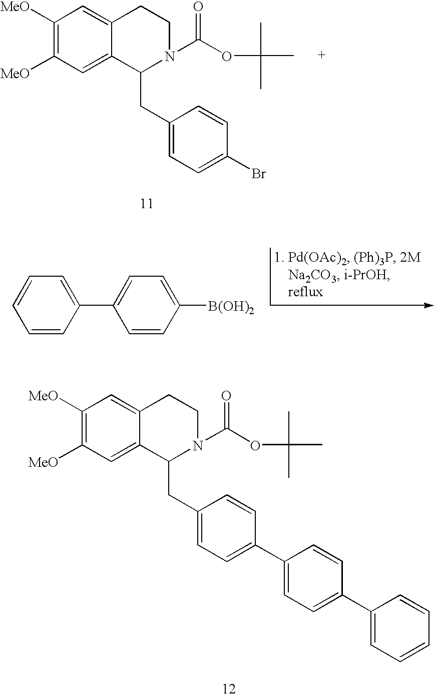

1-[1,1′;4′1″]Terphenyl-4-ylmethyl-1,2,3,4-tetrahydro-isoquinoline-6,7-diol hydrobromide (13) was prepared as follows: To a stirred solution of 0.10 g (0.187 mmol) of 6,7-dimethoxy-1-[1,1′;4′1″]terphenyl-4-ylmethy-1-3,4-dihydro-1H-isoquinoline-2-carboxylic acid tert-butyl ester in 10 ml of anhydrous dichloromethane under argon at −70.degree. C., 1 ml (5 mol. equiv.) of 1M boron tribromide solution in n-hexanes was added. The mixture was allowed to reach room temperature with stirring overnight and then the solvents were evaporated under reduced pressure. The residue was dissolved in 10 ml of methanol and, again, the solvent was evaporated under reduced pressure. The solid residue was mixed with 10 ml of ether, separated on a glass filter funnel, washed with ether (3×20 ml) to afford an off-white solid, 82 mg (90%), mp 262-264° C.

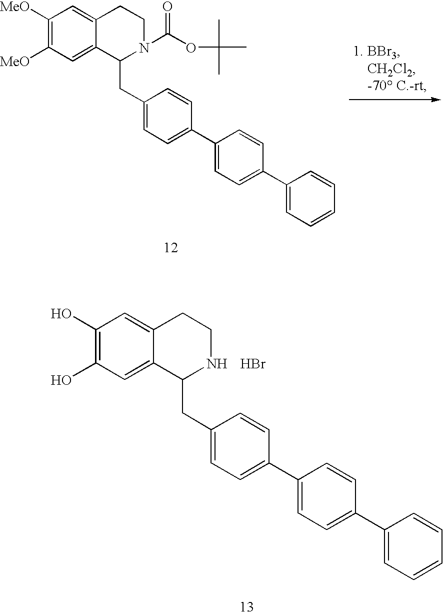

### EXAMPLE 7

### Preparation of the 1-[1,1′;4′1″]Terphenyl-4-ylmethyl-1,2,3,4-tetrahydro-isoquinoline-6,7-diol Hydrobromide Isomers

Stereoisomers of 1-[1,1′;4′1″]Terphenyl-4-ylmethyl-1,2,3,4-tetrahydro-isoquinoline-6,7-diol hydrobromide was prepared according to the reaction scheme below using (+)-menthyl chloroformate, which allows stereoisomers of the intermediate compounds to be separately crystallized using standard conditions.

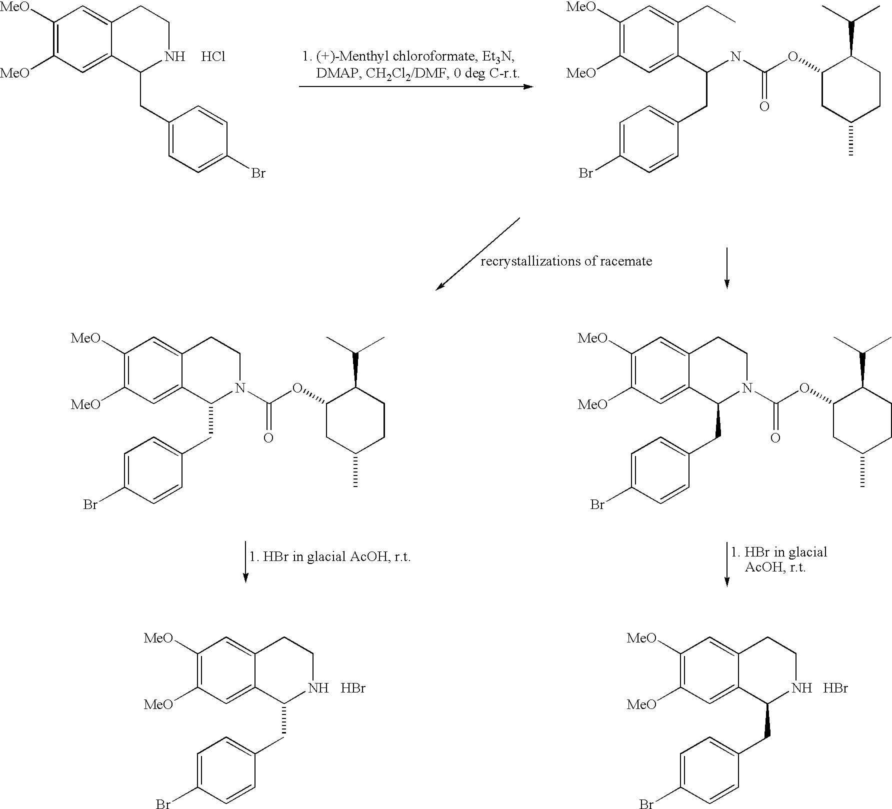

Once the stereoisomers have been separately crystallized, the (+)- and (−)-isomers can be used to prepare substantially pure compounds of the present invention as shown in the two reaction schemes shown below, where each isomer is substantially free of its corresponding isomer.

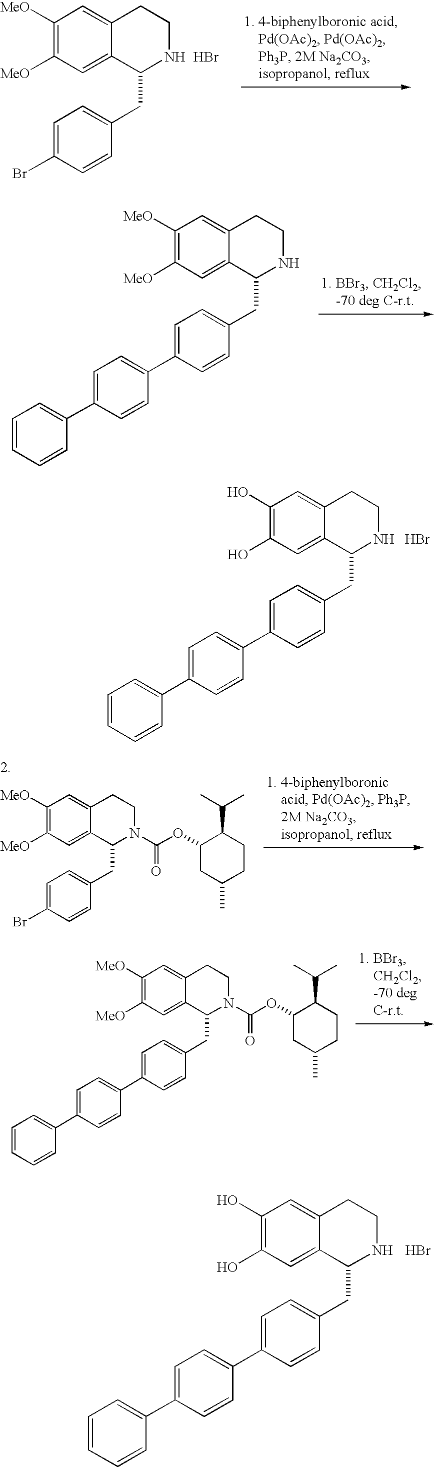

### EXAMPLE 8

### Preparation of 1-(4-benzo[b]thiophen-2-yl-benzyl)-1,2,3,4-tetrahydro-isoquinoline-6,7-diol Hydrobromide

The intermediate 1-(4-Benzo[b]thiophen-2-yl-benzyl)-6,7-dimethoxy-8-methyl-3,4-dihydro-1-H-isoquinoline-2-carboxylic acid tert-butyl ester (21) was prepared as follows: A mixture of 0.462 g (1 mmol) of 1-(4-Bromo-benzyl)-6,7-dimethoxy-3,4-dihydro-1H-isoquinoline-2-carboxylic acid tert-butyl ester and 0.270 g (1.5 mol. equiv.) of thianaphthene-2-boronic acid in 4 ml of isopropanol was stirred under argon at room temperature for 30 min. 1 mg (0.45 mol %) of palladium(II)acetate, 4 mg (1.3 mol %) oftriphenylphosphine, and 0.6 ml (1.2 mol. equiv.) of 2M sodium bicarbonate aqueous solution were added successively and the mixture was refluxed with stirring for 6 hours. The solvent was evaporated under reduced pressure and the residue was partitioned between 50 ml of ethyl acetate and 25 ml of 5% sodium hydroxide aqueous solution. After shaking, the organic layer was washed with water (25 ml) and brine (25 ml), and then dried with sodium sulfate. The solvent was evaporated under reduced pressure and the remaining oil was crystallized by trituration with n-hexanes. The solid was recrystallized using an ethyl acetate\n-hexanes mixture to give 0.41 g (78%) of white solid, mp 137-139° C.

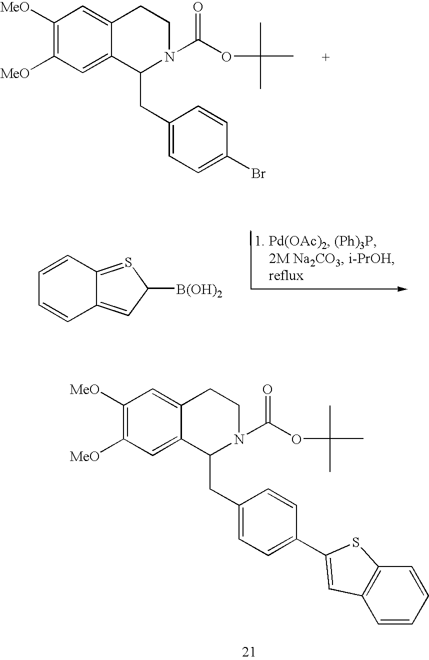

1-(4-Benzo[b]thiophen-2-yl-benzyl)-1,2,3,4-tetrahydro-isoquinoline-6,7-diol hydrobromide (22) was prepared as follows: To a stirred solution of 0.15 g (0.29 mmole) of 1-(4-benzo[b]thiophen-2-yl-benzyl)-6,7-dimethoxy-8-methyl-3,4-dihydro-1-H-isoquinoline-2-carboxylic acid tert-butyl ester in 10 ml of anhydrous dichloromethane under argon at −70° C., 0.9 ml (3 mol. equiv.) of 1M boron tribromide solution in n-hexanes was added. The stirred mixture was allowed to reach room temperature overnight and the solvents were evaporated under reduced pressure. The residue was dissolved in 10 ml of methanol and, again, the solvent was evaporated under reduced pressure. The solid residue was mixed with 10 ml of ether, separated on a glass filter funnel, washed with ether (3×20 ml) to afford an off-white solid, 130 mg (91%), mp 251-254° C.

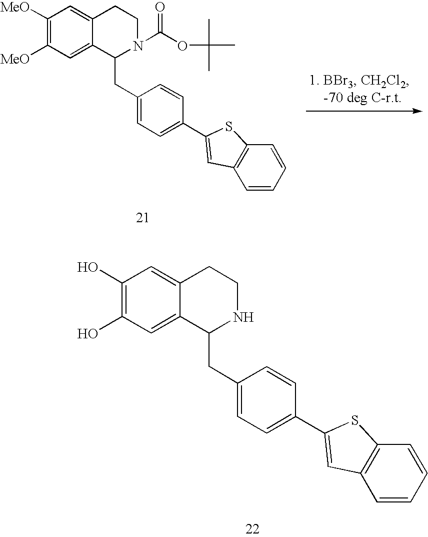

### EXAMPLE 9

### Preparation of 6,7-dimethoxy-1-(3′-methoxy-biphenyl-4-ylmethyl)-1,2,3,4-tetra-hydro-isoquinoline Hydrochloride

The intermediate 2-(4-bromo-phenyl)-N-[2-(3,4-dimethoxy-phenyl)-eth-yl]-acetamide (31) was prepared as follows: A solution of 4-bromo-phenylacetic acid (10.06 g, 46.8 mmol) and oxalyl chloride (101.9 g, 0.8 mol) in 400 mL of benzene was refluxed for 6 h. The solution was evaporated with benzene 3 times and dried in vacuum. A solution of 4-bromo-phenylacetic acid chloride (1.1 mol. equiv.) in CH₂Cl₂ was added to a mixture of 300 mL of 1N NaOH and solution of 3,4-di-methoxy-phenethylamine (6.55 g, 36.1 mmol) in 300 mL of CH₂Cl₂. The reaction mixture was stirred overnight at room temperature. Another portion of acid chloride (0.1 eq.) in 20 mL of CH₂Cl₂ was added and stirred for 1 h. The organic phase was separated, washed with 1N HCl, 1N NaOH, water, dried over Na₂SO₄, filtered and evaporated. A residue was crystallized from EtOAc-hexanes mixture. Yield is 11.54 g (84.5%).

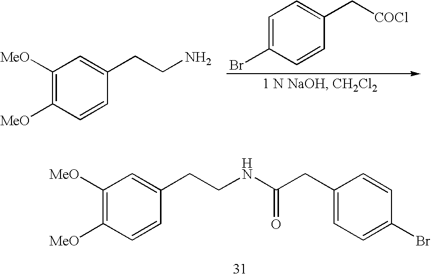

1-(4-bromo-benzyl)-6,7-dimethoxy-1,2,3,4-tetrahydro-isoquinoline oxalate (32) was prepared as follows: A solution of 2-(4-bromo-phenyl)-N-[2-(3,4-dimethoxy-phenyl)-ethyl]-acetamide (1.912 g, 5.05 mmol) and phosphorus oxychloride (24.68 g, 161 mmol) in 50 mL of dry acetonitrile was refluxed for 5 h, cooled, concentrated, evaporated with methanol 3 times and finally dissolved in 50 mL of methanol. Sodium borohydride (3.32 g, 87.8 mmol) was added by small portions. The reaction mixture was stirred overnight at room temperature, concentrated, dissolved in chloroform, washed with 1N NaOH (3 times), dried over Na₂SO₄, concentrated and finally dissolved in methanol. A solution of (COOH)₂.2H₂O (1.28 g, 10.2 mmol) in MeOH was added. The product was crystallized from MeOH-Et₂O mixture. Yield 1.544 g (66%).

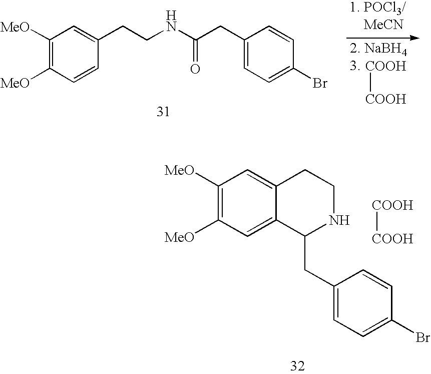

1-(4-bromo-benzyl)-6,7-dimethoxy-1,2,3,4-tetrahydro-isoquinoline hydrochloride (33) was prepared as follows: 1-(4-bromo-benzyl)-6,7-dimethoxy-1,2,3,4-tetrahydro-isoquinoline oxalate (1.24 g, 2.74 mmol) was added to a mixture of 50 mL CH₂Cl₂ and 50 ml of 1N NaOH. The reaction mixture was stirred at room temperature. After dissolving of precipitate, the organic phase was separated, dried over Na₂SO₄, filtered, and evaporated. A residue was dissolved in MeOH and 10 mL of 1M solution of HCl in Et₂O was added. Hydrochloride was crystallized from MeOH-Et₂O mixture. Yield was 0.958 g (87.6%).

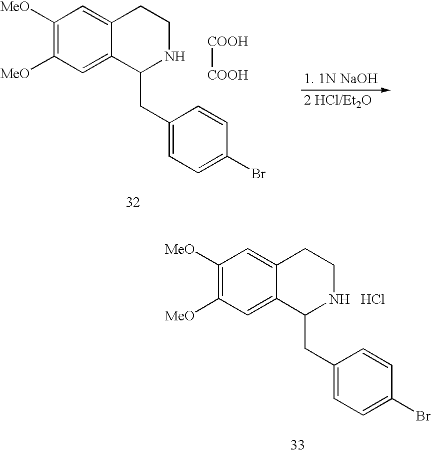

6,7-dimethoxy-1-(3′-methoxy-biphenyl-4-ylmethyl)-1,2,3,4-tetrahydro-isoquinoline hydrochloride (35) was prepared as follows: 1-(4-bromo-benzyl)-6,7-dimethoxy-1,2,3,4-tetrahydro-isoquinoline hydrochloride (0.403 g, 1.01 mmol) was added to a mixture of 20 mL CH₂Cl₂ and 20 ml of 1N NaOH. The reaction mixture was stirred at room temperature. After dissolving the precipitate, the organic phase was separated, dried over Na₂SO₄, filtered, and evaporated. A residue was dissolved in 5 mL i-PrOH and 3-methoxy-phenyl-boronic acid (0.170 g, 1.12 mmol) was added. The resulting solution was stirred for 30 min at room temperature. Pd(OAc)₂ (3 mg, 0.0134 mmol), PPh₃ (8 mg, 0.0305 mmol) and 2N Na₂CO₃ (0.7 mL, 1.4 mmol) were added, the reaction mixture was refluxed for 6 h under Ar and evaporated. A residue was dissolved in chloroform and washed with 1N NaOH. The organic phase was dried under Na₂SO₄, filtered, and evaporated. A residue was dissolved in MeOH, and a solution of oxalic acid dihydrate (0.264 g, 2.08 mmol) was added under reflux. Oxalic acid salt was crystallized from MeOH-Et₂O mixture. Oxalic acid salt was added to a mixture of 20 mL CH₂Cl₂ and 20 ml of 1N NaOH. The reaction mixture was stirred at room temperature. After dissolving of precipitate, the organic phase was separated, dried over Na₂SO₄, filtered, and evaporated. A residue was dissolved in MeOH and 5 mL of 1M solution of HCl in Et₂O was added. Hydrochloride was crystallized from the MeOH-Et₂O mixture. Yield is 0.245 g (56.9%).

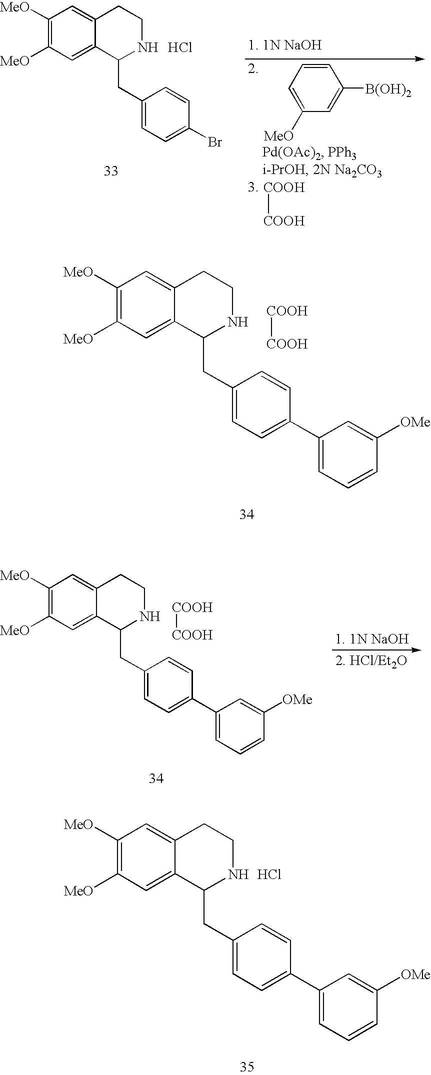

### EXAMPLE 10

### Preparation of 6,7-dimethoxy-1-(4′-methyl-biphenyl-4-ylmethyl)-1,2,3,4-tet-rahydro-isoquinoline Oxalate

6,7-dimethoxy-1-(4′-methyl-biphenyl-4-ylmethyl)-1,2,3,4-tetrahydro-isoquinoline oxalate (36) was prepared as follows: 1-(4-bromo-benzyl)-6,7-dimethoxy-1,2,3,4-tetrahydro-isoquinoline hydrochloride (0.452 g, 1 mmol) was added to a mixture of 20 mL CH.sub.2Cl.sub.2 and 20 ml of 1N NaOH. The reaction mixture was stirred at room temperature. After dissolving of precipitate organic phase was separated, dried over Na₂SO₄, filtered and evaporated. A residue was dissolved in 6 mL i-PrOH. 4-Methyl-phenyl-boronic acid (0.148 g, 1.09 mmol) was added. The resulting solution was stirred for 30 min at room temperature. Pd(OAc)₂ (1 mg, 0.0045 mmol), PPh₃ (7 mg, 0.0267 mmol) and 2N Na₂CO₃ (1 mL, 2 mmol) were added, the reaction mixture was refluxed for 6 h under Ar and evaporated. A residue was dissolved in chloroform and washed with 1N NaOH. The organic phase was dried under Na₂SO₄, filtered and evaporated. A residue was dissolved in MeOH, a solution of oxalic acid dihydrate (0.265 g, 2.1 mmol) was added under reflux. Oxalic acid salt was crystallized from MeOH-Et₂O mixture. Yield was 0.219 g (47.3%).

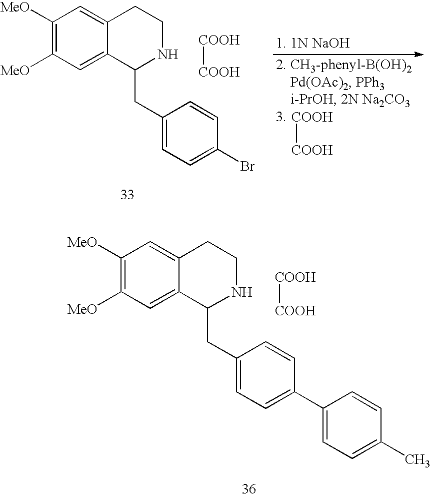

### EXAMPLE 11

### Preparation of 1-(4′-Fluoro-biphenyl-4-ylmethyl) 6,7-dimethoxy-1,2,3,4-tet-rahydro-isoquinoline Oxalate

1-(4′-Fluoro-biphenyl-4-ylmethyl)-6,7-dimethoxy-1,2,3,4-tetrahydro-isoquinoline oxalate (37) was prepared as follows: 1-(4-bromo-benzyl)-6,7-dimethoxy-1,2,3,4-tetrahydro-isoquinoline oxalate (0.452 g, 1 mmol) was added to a mixture of 20 mL CH₂Cl₂ and 20 ml of 1N NaOH. The reaction mixture was stirred at room temperature. After dissolving the precipitate, the organic phase was separated, dried over Na₂SO₄, filtered and evaporated. The residue was dissolved in 6 mL i-PrOH. 4-Fluoro-phenyl-boronic acid (0.153 g, 1.09 mmol) was added. The resulting solution was stirred for 30 min at room temperature. Pd(OAc)₂ (1 mg, 0.0045 mmol), PPh₃ (9 mg, 0.034 mmol) and 2N Na₂CO₃ (1 mL, 2 mmol) were added and the reaction mixture was refluxed for 6 h under Ar and evaporated. Residue was dissolved in chloroform and washed with 1N NaOH. The organic phase was dried under Na₂SO₄, filtered and evaporated. Residue was dissolved in MeOH and a solution of oxalic acid dihydrate (0.259 g, 2.05 mmol) was added under reflux. Oxalic acid salt was crystallized from MeOH-Et₂O mixture. Yield was 0.294 g (62.9%).

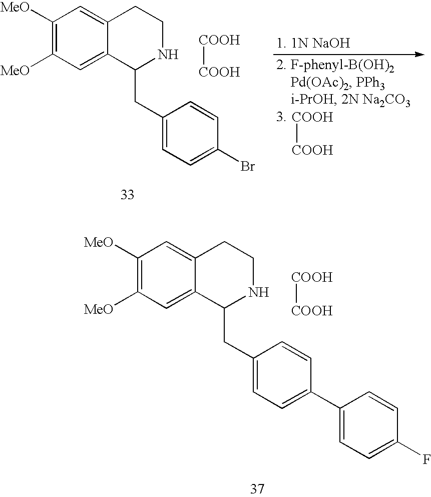

### EXAMPLE 12

### Preparation of 1-Biphenyl-3-ylmethyl-6,7-dimethoxy-1,2,3,4-tetrahydro-isoq-uinoline Oxalate

2-(3-Bromo-phenyl)-N-[2-(3,4-dimethoxy-phenyl)-ethyl]-acetamide (41) was prepared as follows: A solution of 3-bromo-phenylacetic acid (2.0 g, 9.3 mmol) and oxalyl chloride (36.4 g, 0.287 mol) in 80 mL of benzene was refluxed for 6 h. The solution was evaporated with benzene 3 times and dried in vacuum. A solution of 3-bromo-phenylacetic acid (1.1 eq.) in CH₂Cl₂ was added to a mixture of 100 mL of 1N NaOH and a solution of 3,4-di-methoxy-phenethylamine (1.31 g, 7.22 mmol) in 100 mL of CH₂Cl₂. The reaction mixture was stirred overnight at room temperature. Another portion of acid chloride (0.1 eq.) in 10 mL of CH₂Cl₂ was added and stirred for 1 h. The organic phase was separated, washed with 1N HCl, 1N NaOH, water, dried over Na₂SO₄, filtered and evaporated. A residue was crystallized from the EtOAc-hexanes mixtures. Yield was 2.59 g (95%).

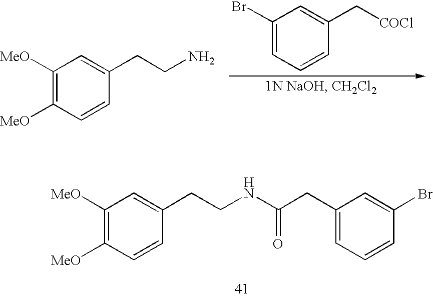

1-(3-bromo-benzyl)-6,7-dimethoxy-1,2,3,4-tetrahydro-isoquinoline oxalate (42) was prepared as follows: A solution of 2-(3-bromo-phenyl)-N-[2-(3,4-dimethoxy-phenyl)-ethyl]-acetamide (2.53 g, 6.7 mmol) and phosphorus oxychloride (24.68 g, 161 mmol) in 120 mL of dry acetonitrile was refluxed for 5 h, cooled, concentrated, evaporated with methanol 3 times, and finally dissolved in 50 mL of methanol. Sodium borohydride (3.23 g, 85.4 mmol) was added by small portions. The reaction mixture was stirred overnight at room temperature, concentrated, dissolved in chloroform, washed with 1N NaOH (3 times), dried over Na₂SO₄, concentrated, and finally dissolved in methanol. A solution of (COOH)₂.2H₂O (1.76 g, 14 mmol) in MeOH was added. The product was crystallized from MeOH-Et₂O mixture. Yield was 1.767 g (58.5%).

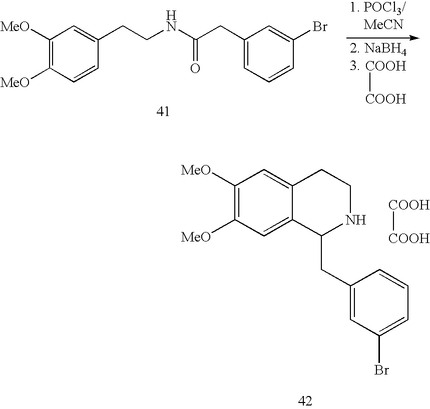

1-biphenyl-3-ylmethyl-6,7-dimethoxy-1,2,3,4-tetrahydro-isoquinoline oxalate (43) was prepared as follows: 1-(3-bromo-benzyl)-6,7-dimethoxy-1,2,3,4-tetrahydro-isoquinoline oxalate (0.910 g, 2.01 mmol) was added to a mixture of 50 mL CH₂Cl₂ and 50 ml of 1N NaOH. The reaction mixture was stirred at room temperature. After dissolving a precipitate organic phase was separated, dried over Na₂SO₄, filtered and evaporated. A residue was dissolved in 10 mL i-PrOH. Phenyl-boronic acid (0.268 g, 2.2 mmol) was added. The resulting solution was stirred for 30 min at room temperature. Pd(OAc)₂ (2 mg, 0.0089 mmol), PPh₃ (11 mg, 0.042 mmol) and 2N Na₂CO₃ (1.3 mL, 2.6 mmol) were added, the reaction mixture was refluxed for 6 h under Ar and evaporated. A residue was dissolved in chloroform and washed with 1N NaOH. The organic phase was dried under Na₂SO₄, filtered and evaporated. A residue was dissolved in MeOH and a solution of oxalic acid dihydrate (0.510 g, 4.05 mmol) was added under reflux. Oxalic acid salt was crystallized from the MeOH-Et₂O mixture. Yield was 0.558 g (61.7%)

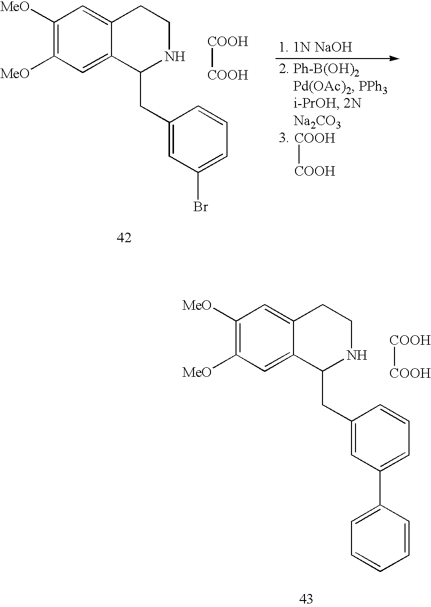

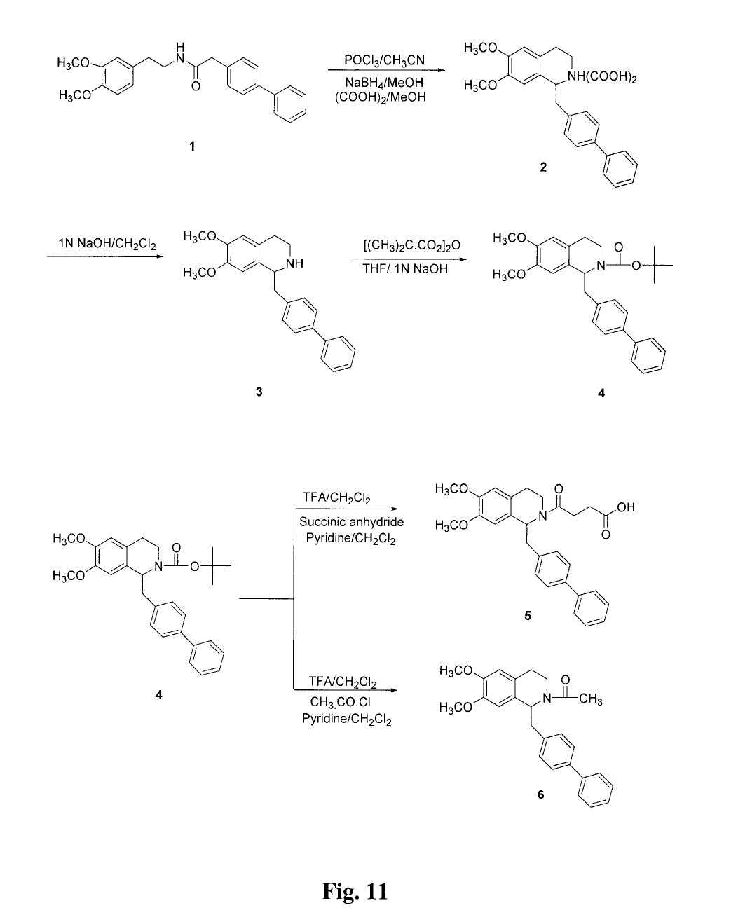

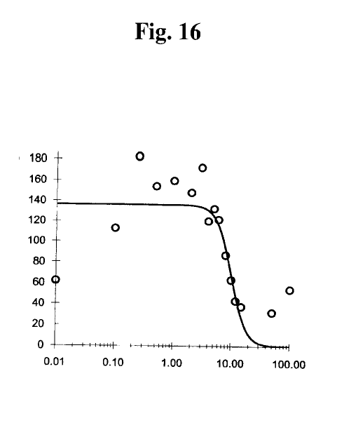

Compounds where R₈ is 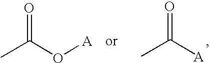
where A is substituted or unsubstituted alkyl, alkoxy, alkyloxy, or arylalkyl, may be synthesized as described below. Numbers refer to compounds of Table 1 and FIGS. 11-20.

The inventors have discovered that the carbamates, where R₈ is 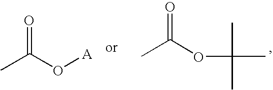
and the amides, where R₈ is 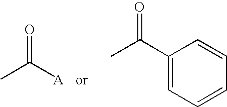
(benzamides), exhibit significant activity as anti-glioma agents, are highly selective, and exhibit very low toxicity to normal astrocytes.

The synthesis of 1-Biphenyl-4-ylmethyl-6,7-dimethoxy-3,4-dihydro-1H-isoquinoline-2-carboxylic acid tert-butyl ester 4, 4-(1-Biphenyl-4-ylmethyl-6,7-dimethoxy-3,4-dihydro-1H-isoquinolin-2-yl)-4-oxobutyric acid 5, and 1-(1-Biphenyl-4-ylmethyl-6,7-dimethoxy-3,4-dihydro-1H-isoquinolin-2-yl)ethanone 6 is outlined in FIG. 11. 1-Biphenyl-4-ylmethyl-6,7-dimethoxy-1,2,3,4-tetrahydroisoquinoline oxalate salt 2 was synthesized from Bischler-Neperski acclimation of the amide 2-Biphenyl-4-yl-N-[2-(3,4-dimethoxyphenyl)ethyl]acetamide 1 using POCl₃ in anhydrous acetonitrile followed by reduction with NaBH₄. The salt formation was achieved with oxalic acid in CHCl₃/MeOH. The compound 2 was treated with 1N NaOH in dichloromethane to get free amine 3, and this amine was protected with di-tert-butyl dicarbonate in the presence of 1N NaOH in THF to afford 1-Biphenyl-4-ylmethyl-6,7-dimethoxy-3,4-dihydro-1H-isoquinoline-2-carboxylic acid tert-butyl ester 4. The compound 4 was treated with trifluoro acetic acid in dichloromethane to give free amine 3, which was further coupled with succinic anhydride in the presence of pyridine in dichloromethane to obtain compound 5. 1-(1-Biphenyl-4-ylmethyl-6,7-dimethoxy-3,4-dihydro-1H-isoquinolin-2-yl)ethanone 6 was prepared from the reaction of compound 4 with trifluoroacetic acid in dichloromethane, followed by acetyl chloride in dichloromethane using pyridine as base.

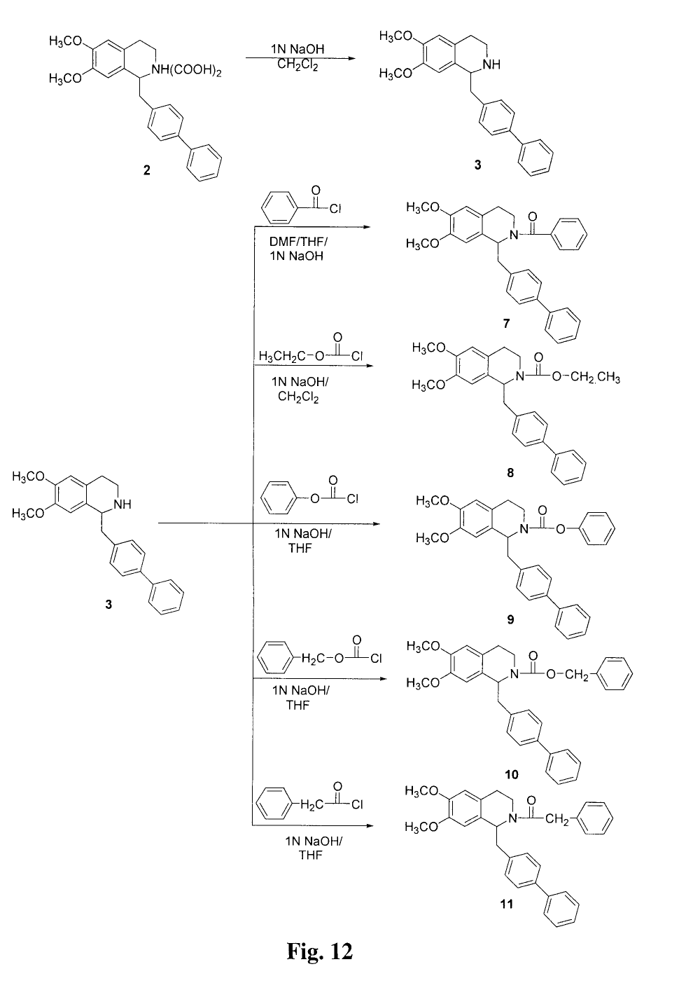

1-Biphenyl-4-ylmethyl-6,7-dimethoxy-1,2,3,4-tetrahydroisoquinoline 3 was obtained by treating the compound 2 with 1N NaOH in dichloromethane, which was further coupled with benzoyl chloride in DMF, THF, and 1N NaOH aqueous solution to obtain (1-Biphenyl-4-ylmethyl-6,7-dimethoxy-3,4-dihydro-1H-isoquinolin-2-yl)phenyl-methanone 7. Reaction of compound 3 with ethyl chloroformate in the presence of 1N NaOH in dichloromethane gave 1-Biphenyl-4-ylmethyl-6,7-dimethoxy-3,4-dihydro-1H-isoquinoline-2-carboxylic acid ethyl ester 8. 1-Biphenyl-4-ylmethyl-6,7-dimethoxy-3,4-dihydro-1H-isoquinoline-2-carboxylic acid phenyl ester 9, 1-Biphenyl-4-ylmethyl-6,7-dimethoxy-3,4-dihydro-1H-isoquinoline-2-carboxylic acid benzyl ester 10, 1-(1-Biphenyl-4-ylmethyl-6,7-dimethoxy-3,4-dihydro-1H-isoquinolin-2-yl)-2-phenyl-ethanone 11 were synthesized by reacting free amine 3 in THF with Phenyl chloroformate, benzyl chloroformate, and phenyl acetylchloride using 1N NaOH (FIG. 12).

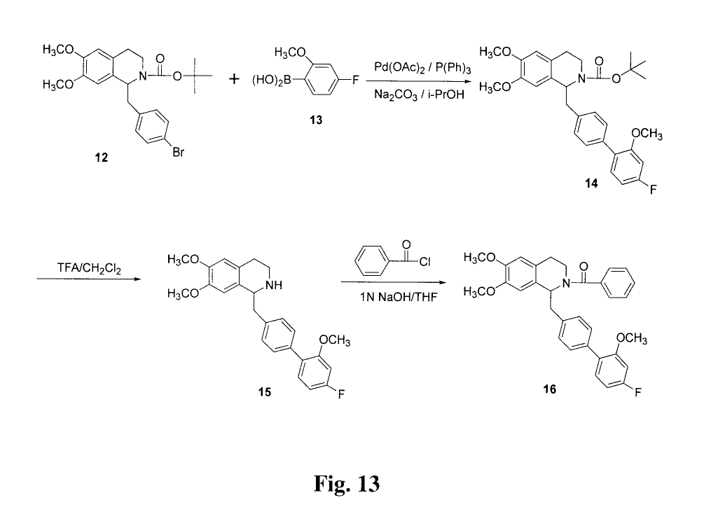

1-(4′-Fluoro-2′-methoxybiphenyl-4-ylmethyl)-6,7-dimethoxy-3,4-dihydro-1H-isoquinoline-2-carboxylic acid tert-butyl ester 14 was synthesized by the Suzuki coupling of 1-(4-Bromobenzyl)-6,7-dimethoxy-3,4-dihydro-1H-isoquinoline-2-carboxylic acid tert-butyl ester 12¹ with 4-Fluoro-2-methoxyphenylboronic acid using Palladium(II)acetate, triphenylphosphine, and Na₂CO₃ in anhydrous isopropanol. Compound 14 was treated with trifluoroacetic acid in dichloromethane to furnish free amine, 1-(4′-Fluoro-2′-methoxybiphenyl-4-ylmethyl)-6,7-dimethoxy-1,2,3,4-tetrahydroisoquinoline 15, which was further coupled with benzoyl chloride in the presence of 1N NaOH in THF to afford [1-(4′-Fluoro-2′-methoxybiphenyl-4-ylmethyl)-6,7-dimethoxy-3,4-dihydro-1H-isoquinolin-2-yl]phenylmethanone 16 (FIG. 13).

All the reagents and solvents were purchased from Aldrich, Lancaster, and used without further purification. The reactions were performed under nitrogen atmosphere. Proton NMR spectra were recorded on a Bruker ARX 300 spectrometer (300 MHz) using DMSO-d₆ and CDCl₃ as solvents, and spectral data were consistent with assigned structures. Chemical shift values were reported as parts per million (δ), coupling constants (J) are given in Hz, and splitting patterns are designated as follows: s, singlet; d, doublet; t, triplet; q, quartet; m, multiplet. Mass spectra were collected on a Brucker ESQUIRE electrospray/ion trap instrument (Brucker Dalton, Germany) in the positive and negative modes. Elemental analysis was done by Atlantic Microlab Inc., Norcross, Ga., and values obtained were within ±0.4% of the theoretical values. Routine thin-layer chromatography (TLC) was performed on silica gel plates (Analtech, Inc., 250 microns). Flash chromatography was conducted on silica gel (Merck, grade 60, 230-400 mesh).

### 1-Biphenyl-4-ylmethyl-6,7-dimethoxy-1,2,3,4-tetrahydroisoquinoline Oxalate Salt (2)

Phosphorus oxychloride (30.6 g, 199.8 mmol) was added to a stirred solution of 2-Biphenyl-4-yl-N-[2-(3,4-dimethoxyphenyl)ethyl]acetamide 1 (2.5 g, 6.658 mmol) in anhydrous acetonitrile (100 mL) and refluxed for 6 h. The reaction mixture was concentrated under reduced pressure. Methanol was added to the reaction mixture, and again concentrated the solvent under reduced pressure. Sodium borohydride (3.778 g, 99.8mmol) was added portion wise to above residue in methanol (30 mL) at 0° C., and the reaction mixture was stirred over night at room temperature. The solvent was removed under reduced pressure, and the oily mass was dissolved in CHCl₃, washed with 1N NaOH solution (60 mL), water and dried over Na₂SO₄. The solvent was removed under reduced pressure, and the residue was dissolved in CHCl₃. A solution of oxalicacid dihydrate (1.679 g, 13.317 mmol) in methanol was added to above solution with stirring at room temperature followed by addition of ether. The mixture was stirred for 2 h at the same temperature and kept in the refrigerator overnight. The solid was filtered and washed with mother liquor, finally with ether, and air-dried over night to afford white solid 2 (2.165 g, 72% ). ¹H NMR (300 MHz, DMSO-d6) δ 7.70-7.67 (m, 4H, ArH), 7.48-7.42 (m, 5H, ArH), 6.80(s, 1H, ArH), 6.51(s, 1H, ArH), 4.65(bs, 1H, CH), 3.74(s, 3H, OCH₃), 3.53(s, 3H, OCH₃), 3.50-3.35 (m, 2H, CH₂), 3.30-3.10 (m, 2H, CH₂), 2.98(bs, 2H, CH₂). MS (ES+) m/z 360 [M-(COOH)₂+H]⁺.

### 1-Biphenyl-4-ylmethyl-6,7-dimethoxy-3,4-dihydro-1H-isoquinoline-2-carboxylic Acid Tert-Tbutyl Ester (4)

To a solution of 1-Biphenyl-4-ylmethyl-6,7-dimethoxy-1,2,3,4-tetrahydroisoquinoline oxalate salt 2 (1.5 g, 3.337 mmol) in dichloromethane (100 mL) was added 1N NaOH (60 mL), and the mixture was stirred at room temperature for 2 h. The two layers were separated, and the aqueous layer was extracted with dichloromethane (2×50 mL) and dried over MgSO₄. The solvents were removed under reduced pressure to provide 1-Biphenyl-4-ylmethyl-6,7-dimethoxy-1,2,3,4-tetrahydroisoquinoline 3 as yellow oil. A solution of di-t-butyl dicarbonate (1.1 g, 5.005 mmol) in tetrahydro furan (10 mL) was added to a stirred solution of mixture of above oil in tetrahydro furan (30 mL) and 1N NaOH aqueous solution (10 mL) at 0° C. The reaction mixture was stirred over night at room temperature, and the solvents were concentrated under reduced pressure. The residue was diluted with water, and extracted with dichloromethane. The organic layer was dried over MgSO₄ and solvent was evaporated under reduced pressure. Crude residue was purified by flash column chromatography using MeOH—CHCl₃ (0.1:99.9 to 1.0:99.0 v/v), and the obtained oily mass was recrystallized with ethyl acetate-ether to obtain white crystals 4 (1.25 g, 81%). In the NMR spectrum, two sets of peaks were appeared in the ratio of 1:0.37. ¹H NMR (300 MHz, DMSO-d6) δ 7.70-7.54 (m, 6H, ArH), 7.48-7.43 (m, 3H, ArH), 7.37-7.23 (m, 4H, ArH), 6.86 (s, 1H, ArH), 6.71 (s, 1H, ArH), 5.23 (s, 1H, —CH—), 5.13-5.08 (m, 1H, —CH—), 3.73 (s, 8H, —OCH₃), 3.31-3.22 (m, 2H, —CH₂), 3.12-2.98 (m, 3H, —CH₂), 2.75-2.65 (m, 3H, —CH₂), 1.30 (s, 3H, CH₃), 1.09 (s, 9H, 3×CH₃). MS (ES+) m/z 482 (M+Na)⁺.

### 4-(1-Biphenyl-4-ylmethyl-6,7-dimethoxy-3,4-dihydro-1H-isoquinolin-2-yl)-4-oxobutyric Acid (5)

Trifluoroacetic acid (1.240 g, 10.879 mmol) was added to a stirring solution of 1-Biphenyl-4-ylmethyl-6,7-dimethoxy-3,4-dihydro-1H-isoquinoline-2-carboxylic acid tert-butylester 4 (0.500 g, 1.088 mmol) in dichloromethane (15 mL) at 0° C. and the mixture was stirred for 4 h allowing the solution to warm to ambient temperature. The reaction mixture was concentrated to dryness under reduced pressure, and the residue was extracted with ethyl acetate. The organic layer was washed with saturated aqueous NaHCO₃ followed by water, and dried over anhydrous Na₂SO₄ The solvents were removed in vacuum to yield 1-Biphenyl-4-ylmethyl-6.7-dimethoxy-1,2,3,4-tetrahydroisoquinoline 3 as yellow colored oil. The oil was dissolved in dichloromethane (10 mL) and cooled to 0° C. A solution of succinic anhydride (0.109 g, 1.088 mmol) in dichloromethane (5 mL) and pyridine (0.215 g, 2.720 mmol) were added, and the mixture was stirred overnight at room temperature. The reaction mixture was extracted with dichloromethane, the organic phase was washed with 10% aqueous HCl (2×5 mL), water and dried with MgSO₄ The solvents were evaporated under reduced pressure. The residue was dissolved in ether (2×10 mL), and ether was evaporated to get pale yellow solid 5 (0.150 g, 30%). The ¹H NMR spectrum showed two sets of peaks in the ratio of 1:1. ¹H NMR (CDCl₃) δ 11.11-10.07 (bs, 2H, —COOH), 7.61-7.33 (m,15H, ArH), 7.24-7.11 (m, 3H, ArH), 6.66 (s, 1H, ArH), 6.63 (s, 1H, ArH), 6.51 (s, 1H, ArH), 6.13 (s, 1H, ArH), 5.70-5.63 (m, 1H, —CH—), 4.92-4.88 (m, 1H, —CH—), 3.84 (d, J=6.0 Hz, 6H, 2×—OCH₃), 3.81 (s, 3H, OCH₃), 3.72-3.70 (m, 2H, —CH₂), 3.54 (s, 3H, —OCH₃), 3.38-3.18(m, 4H, 2×—CH₂), 3.03-2.71(m, 10H, 5×—CH₂), 2.50-2.24 (m, 4H, 2×—CH₂). MS (ES−) m/z 458 (M−H)⁻. Analysis calculated for C₂₈H₂₉NO₅.

### 1-(1-Biphenyl-4-ylmethyl-6,7-dimethoxy-3,4-dihydro-1H-isoquinolin-2-yl)ethanone (6)

Trifluoroacetic acid (2.481 g, 21.758 mmol) was added to a stirring solution of 1-Biphenyl-4-ylmethyl-6,7-dimethoxy-3,4-dihydro-1H-isoquinoline-2-carboxylic acid tert-butylester 4 (0.500 g, 1.088 mmol) in dichloromethane (15 mL) at 0° C. and the mixture was stirred for 3 h allowing the solution to warm to ambient temperature. The solvents were removed in vacuo, and the residue was extracted with ethyl acetate. The organic phase was washed with saturated aqueous NaHCO₃ followed by water, and dried over anhydrous Na₂SO₄ The solvent was removed under reduced pressure, and then dried to obtain 1-Biphenyl-4-ylmethyl-6,7-dimethoxy-1,2,3,4-tetrahydroisoquinoline 3 as yellow colored oil. Acetic anhydride (0.122 g, 1.197 mmol) and pyridine (0.215 g, 2.720 mmol) were added to the oil, which was dissolved in dichloromethane (15 mL) at 0° C. The reaction mixture was stirred overnight at room temperature, and diluted with dichloromethane and water. Two layers were separated and the organic phase was washed with saturated aqueous NaHCO₃ solution to remove acetic acid, and dried with anhydrous MgSO₄. The solvents were evaporated under reduced pressure. The residue was dissolved in ether (3×10 mL), and again the solvent was removed under reduced pressure to give a white solid 6 (0.175 g, 40%). The proton NMR spectrum showed two sets of peaks in the ratio of 1:1. ¹H NMR (DMSO-d₆) δ 7.70-7.63 (m, 6H, ArH), 7.56 (d, J=8.1 Hz, 2H, ArH), 7.49-7.31 (m, 8H, ArH), 7.22 (d, J=8.1 Hz, 2H, ArH), 6.91 (s, 1H, ArH), 6.72 (s, 2H, ArH), 6.53 (s, 1H, ArH), 5.61 (t, J=6.9 Hz, 1H, —CH—), 5.01-4.96 (m, 1H, —CH—), 3.72 (d, J=3.9 Hz, 9H, 3×—OCH₃), 3.56 (s, 3H, —OCH₃), 3.24-3.04 (m, 6H, 3×—CH₂), 2.86-2.66 (m, 6H, 3×—CH₂), 2.00 (s, 3H, —CH₃), 1.48 (s, 3H, —CH₃). MS (ES+) m/z 424 (M+Na)⁺. Analysis calculated for C₂₆H₂₇NO₃.

### (1-Biphenyl-4-ylmethyl-6,7-dimethoxy-3,4-dihydro-1H-isoquinolin-2-yl)phenyl-methanone (7)

A 1N NaOH solution (50 mL) was added to a solution of 1-Biphenyl-4-ylmethyl-6,7-dimethoxy-1,2,3,4-tetrahydroisoquinoline oxalate salt 2 (0.800 g, 1.780 mmol) in dichloromethane (50 mL), and this biphasic solution mixture was stirred at room temperature for 2 h. Two layers were separated, the aqueous layer was extracted with dichloromethane (2×25 mL), and the organic layers were dried over MgSO₄. The solvents were evaporated under reduced pressure, and then dried for 6 h under high vacuum to provide 1-Biphenyl-4-ylmethyl-6,7-dimethoxy-1,2,3,4-tetrahydroisoquinoline 3 as a yellow colored oil (0.350 g, 55%). To a stirred solution of the oil (0.350 g, 0.974 mmol) in dry THF (5 mL) and dry DMF (5 mL)were successively added 1 N NaOH (2 mL) and benzoyl chloride (0.164 g, 1.168 mmol). The reaction mixture was stirred overnight at room temperature. The mixture was quenched with water and extracted with ethyl acetate. The organic solvents were washed with water followed by brine and dried over anhydrous Na₂SO₄ The solvents were evaporated under reducing conditions, and the crude residue was purified by flash column chromatography using MeOH—CHCl₃ (0.1:99.9 to 2.0:98.0 v/v) to obtain a white solid 7 (0.62 g, 25.0%). [The starting material, free amine 2 was recovered after purification (0.158 g, 45%)].

In the ¹H NMR spectrum, two sets of peaks were observed in the ratio of 1:0.76. ¹H NMR (CDCl₃) δ 7.60-7.21 (m, 22H, ArH), 6.97-6.87 (m, 3H, ArH), 6.68 (s, 1H, ArH), 6.58 (s, 1H, ArH), 6.33 (s, 1H, ArH), 5.94 (t, J=6.6 Hz, 1H, —CH—), 4.93-4.85 (m, 1H, —CH—), 3.87 (d, J=7.2 Hz, 5H, —OCH₃), 3.65 (s, 6H, 2×—OCH₃), 3.52-3.09 (m, 6H, 3×—CH₂), 2.98-2.55 (m, 5H, —CH₂). MS (ES+) m/z 486 (M+Na)⁺. Anal. Calcd. for C₃₁H₂₉NO₃.

### 1-Biphenyl-4-ylmethyl-6,7-dimethoxy-3,4-dihydro-1H-isoquinoline-2-carboxylic Acid Ethyl Ester (8)

1N NaOH solution (50 mL) was added to a solution of 1-Biphenyl-4-ylmethyl-6,7-dimethoxy-1,2,3,4-tetrahydroisoquinoline oxalate salt 2 (0.800 g, 1.780 mmol) in dichloromethane (50 mL), and this biphasic solution mixture was stirred at room temperature for 2 h. The two layers were separated and the aqueous layer was extracted with dichloromethane (2×25 mL) and dried with anhydrous MgSO₄. The solvents were removed under reduced pressure, which yielded 1-Biphenyl-4-ylmethyl-6,7-dimethoxy-1,2,3,4-tetrahydroisoquinoline 3 as yellow colored oil. This oil was used for the next step without further purification. Ethyl chloroformate (0.386 g, 3.559 mmol) and 1N NaOH (8 mL) were added to the oil in dichloromethane (15 mL) at 0° C. The reaction mixture was stirred overnight at room temperature. The mixture was diluted with water and the two layers were separated. The aqueous layer was washed with dichloromethane, and the organic phase was dried over anhydrous MgSO₄ The solvents were evaporated under reduced pressure, and the oily residue was dissolved in a mixture of dichloromethane and ether (1:1) (4×10 mL), and again removed the solvents under reduced pressure to provide an off-white solid 8 (0.269 g, 35%). The ¹H NMR spectrum showed two sets of peaks in the ratio of 1:0.6. ¹H NMR (DMSO-d₆) δ 7.72-7.56 (m, 8H, ArH), 7.48-7.43 (m, 4H, ArH), 7.36-7.20 (m, 4H, ArH), 6.85 (s, 1H, ArH), 6.71 (s, 1H, ArH), 5.26 (t, J=6.8 Hz, 1H, —CH—), 5.11-5.20 (m, 1H, —CH—), 4.10-4.05 (m, 2H, —CH₂), 3.96 (q, J=6.3 Hz, J=6.3 Hz, 2H, —CH₂), 3.71 (d, J=5.4 Hz, 9H, 3×—OCH₃), 3.14-2.93 (m, 4H, 2×—CH₂), 2.81-2.57 (m, 5H, —CH₂), 1.10 (t, J=6.9 Hz, 2H, —CH₃), 0.85 (t, J=6.9 Hz, 3H, —CH₃). MS (ES+) m/z 454 (M+Na)⁺. Anal. calcd. for C₂₇H₂₉NO₄.

### 1-Biphenyl-4-ylmethyl-6,7-dimethoxy-3,4-dihydro-1H-isoquinoline-2-carboxylic Acid Phenyl Ester (9)

To a solution of 1-Biphenyl-4-ylmethyl-6,7-dimethoxy-1,2,3,4-tetrahydroisoquinoline oxalate salt 2 (0.800 g, 1.780 mmol) in dichloromethane (50 mL), 1N NaOH solution (50 mL) was added, and mixture was stirred at room temperature for 2 h. The two layers were separated, the aqueous layer extracted with dichloromethane (2×25 mL), and the combined organic layers were dried over MgSO₄. The solvents were removed under reduced pressure to yield 1-Biphenyl-4-ylmethyl-6,7-dimethoxy-1,2,3,4-tetrahydroisoquinoline 3 as yellow colored oil, which was used for the next step without further purification. Phenyl chloroformate (0.418 g, 2.670 mmol) was added drop-wise to a stirred mixture of the oil in tetrahydrofuran (15 mL) and 1N NaOH solution (8 mL) at 0° C. The reaction mixture was stirred over night at room temperature and the solvents were removed under reduced pressure. The residue was dissolved in water, and extracted with ethyl acetate. The organic phase was dried over Na₂SO₄ and the solvents were evaporated under reduced pressure. Crude oily residue was recrystallized using a dichloromethane-ether mixture to give white solid 9 (0.486 g, 57%). Two sets of peaks appeared in the proton NMR in the ratio of 1:0.6. ¹H NMR (CDCl₃) δ 7.59-7.28 (m, 15H, ArH), 7.22-7.17 (m, 4H, ArH), 7.12-7.04 (m, 2H, ArH), 6.68-6.65 (m, 3H, ArH), 6.50 (s, 1H, ArH), 6.19 (s, 1H, ArH), 5.46 (t, J=5.7 Hz, 1H, —CH—), 5.35 (t, J=6.3 Hz, 1H, —CH—), 3.88 (d, J=5.1 Hz, 2H, —OCH₃), 3.78 (s, 3H, —OCH₃), 3.60 (s, 2H, —OCH₃), 3.53-3.12 (m, 5H, —CH₂), 3.09-2.71 (m, 5H, 3×—CH₂). MS (ES+) m/z 502 (M+Na)⁺. Anal. calcd. for C₃₁H₂₉NO₄.

### 1-Biphenyl-4-ylmethyl-6,7-dimethoxy-3,4-dihydro-1H-isoquinoline-2-carboxylic Acid Benzyl Ester (10)

This compound was synthesized as described for compound 9 using compound 2 (0.800 g, 1.780 mmol), benzyl chloroformate (0.455 g, 2.670 mmol), and 1N NaOH solution (8 mL). The crude residue was recrystallized with benzene-pet.ether to get 9 white powder (0.550 g, 62.6% ) M.P. The ¹H NMR spectrum showed two sets of peaks in the ratio of 1:1. ¹H NMR (CDCl₃) δ 7.61-7.54 (m, 4H, ArH), 7.49-7.41 (m, 9H, ArH), 7.36-7.29 (m, 10H, ArH), 7.17-7.09 (m, 5H, ArH), 6.60 (d, J=9.3 Hz, 2H, ArH), 6.27 (s, 1H, ArH), 6.15 (s, 1H, ArH), 5.33 (t, J=6.3 Hz, 1H, —CH—), 5.27-5.16 (m, 3H, —CH— & —CH₂), 5.02 (d, J=12.0 Hz, 1H, —CH₂), 4.90 (d, J=12.0 Hz, 1H, —CH₂), 3.85 (d, J=3.0 Hz, 6H, 2×—OCH₃), 3.66 (s, 3H, —OCH₃), 3.57 (s, 3H, —OCH₃), 3.50-3.35 (m, 2H, CH₂), 3.27-3.12 (m, 2H, —CH₂), 3.06-2.57 (m, 8H, 4×—CH₂). MS (ES+) m/z 516 (M+Na)⁺. Anal. calcd. for C₃₂H₃₁NO₄.

### 1-(1-Biphenyl-4-ylmethyl-6,7-dimethoxy-3,4-dihydro-1H-isoquinolin-2-yl)-2-phenyl-ethanone (11)

This compound was synthesized as described for compound 9 using compound 2 (0.800 g, 1.780 mmol), phenyl acetylchloride (0.550 g, 3.559 mmol), and 1N NaOH solution (8 mL). The crude residue was crystallized with benzene-pet.ether to afford 11 as white solid (0.480 g, 56.5%). In the proton NMR spectrum, two sets of peaks were observed in the ratio of 1:0.6. ¹H NMR (CDCl₃) δ 7.62-7.55 (m, 5H, ArH), 7.47-7.38 (m, 5H, ArH), 7.36-7.23 (m, 4H, ArH), 7.21-7.13 (m, 7H, ArH), 6.90-6.88 (m, 1H, ArH), 6.63 (s, 1H, ArH), 6.54 (s, 1H, ArH), 6.34 (d, J=1.2 Hz, 1H, ArH), 6.17 (d, J=1.5 Hz, 1H, ArH), —CH—), 5.76 (t, J=6.3 Hz, 1H, —CH—), 4.98-4.94 (m, 1H, —CH—), 3.87 (d, J=1.8 Hz, 2H, —CH₂Ph), 3.83 (d, J=1.5 Hz, 3H, —OCH₃), 3.78 (s, 4H, —OCH₃), 3.75 (d, J=1.8 Hz, 1H, —CH₂Ph), 3.57 (d, J=1.8 Hz, 3H, —OCH₃), 3.49-3.41 (m, 2H, —CH₂), 3.35-2.89 (m, 7H, —CH₂), 2.75-2.58 (m, 1H, CH₂). MS (ES+) m/z 500 (M+Na)⁺. Anal. calcd. for C₃₂H₃₁NO₃

### 1-(4′-Fluoro-2′-metioxybiphenyl-4-ylmethyl)-6,7-dimethoxy-3,4-dihydro-1H-isoquinoline-2-carboxylic Acid tert-butyl Ester (14)

A mixture of 1-(4-Bromobenzyl)-6,7-dimethoxy-3,4-dihydro-1H-isoquinoline-2-carboxylic acid tert-butyl ester 12 (0.500 g, 1.081 mmol), Palladium(II)acetate (0.007 g, 3 mol %), and triphenylphosphine (0.017 g, 6 mol %) in anhydrous isopropanol (10 mL) was stirred under dry conditions at room temperature for 30 min. To this mixture, 4-Fluoro-2-methoxyphenylboronic acid (0.276 g, 1.622 mmol), and Na₂CO₃ (0.344 g, 3.244 mmol) were added successively, and the mixture was refluxed with stirring overnight. The solvent was concentrated under reduced pressure, the residue was partitioned between ethyl acetate (50 mL), and 5% NaOH aqueous solution (25 mL). Two layers were separated, and the aqueous layer was washed with ethyl acetate (10 mL). The organic layers were washed with water followed by brine, and dried with Na₂SO₄. The solvent was evaporated under reduced pressure, and the crude residue was purified by flash column chromatography using ethyl acetate-hexane (5:95 to 50:50 v/v), to afford white solid 14 (0.406 g, 74%). In the ¹H NMR spectrum, two sets peaks were appeared in the ratio of 1:0.45. ¹H NMR (CDCl₃) δ 7.42-7.35 (m, 3H, ArH), 7.24-7.17 (m, 4H, ArH), 6.76-6.61 (m, 4H, ArH), 6.37 (s, 1H, ArH), 6.13 (s, 1H, ArH), 5.31 (t, J=7.5 Hz, 1H, —CH—), 5.17 (t, J=7.5 Hz, 1H, —CH—), 3.88 (s, 5H, —OCH₃), 3.79 (s, 5H, —OCH₃), 3.74 (s, 3H, —OCH₃), 3.46-3.28 (m, 2H, —CH₂), 3.23-3.01 (m, 2H, —CH₂), 3.01-2.82 (m, 4H, 2×—CH₂), 1.45 (s, 4H, —OCH₃), 1.31 (s, 9H, 3×—OCH₃).MS (ES+) m/z 530 (M+Na)⁺. Anal. calcd. for C₃₀H₃₄FNO₅.

### [1-(4′-Fluoro-2′-methoxybiphenyl-4-ylmethyl)-6,7-dimethoxy-3,4-dihydro-1H-isoquinolin-2-yl]phenylmethanone (16)

To a solution of 1-(4′-Fluoro-2′-methoxybiphenyl-4-ylmethyl)-6,7-dimethoxy-3,4-dihydro-1H-isoquinoline-2-carboxylic acid tert-butyl ester 14 (0.250 g, 0.493 mmol) in dichloromethane (10 mL) was added trifluoroacetic acid (1.123 g, 9.850 mmol) at 0° C., and the mixture was stirred for 3 h, allowing the solution to warm to room temperature. The solvents were evaporated under reduced pressure, and the residue was extracted with ethyl acetate. The organic layer was washed with saturated aqueous NaHCO₃ followed by water, and dried with anhydrous Na₂SO₄ The solvent was removed under reduced pressure to obtain 1-(4′-Fluoro-2′-methoxybiphenyl-4-ylmethyl)-6,7-dimethoxy-1,2,3,4-tetrahydroisoquinoline 15 as yellow oil. This oil was used without further purification. Benzoyl chloride (0.277 g, 1.970 mmol) was added dropwise to a stirred mixture of 15 in tetrahydrofuran (10 mL), and 1N NaOH aqueous solution (2 mL) at 0° C. The mixture was stirred over night at room temperature, and the solvents were evaporated under reduced pressure. The residue was dissolved in water, and extracted with ethyl acetate. The organic phase was dried over Na₂SO₄, and the solvent was removed under reduced pressure. The crude residue was recrystallized using benzene-pet.ether to get 16 as white solid (0.150 g, 59.5%). The proton NMR spectrum showed two sets peaks in the ratio of 1:1. ¹H NMR (CDCl₃) δ 7.64-7.47 (m, 1H, ArH), 7.45-7.29 (m, 13H, ArH), 7.23-7.16 (m, 3H, ArH), 6.94-6.88 (m, 3H, ArH), 6.76-6.67 (m, 5H, ArH), 6.58 (s, 1H, ArH), 6.28 (s, 1H, ArH), 6.13 (s, 1H, ArH), 5.94 (t, J=6.9 Hz, 1H, —CH—), 4.96-4.82 (m, 1H, —CH—), 3.87 (d, J=7.5 Hz, 6H, 2×—OCH₃), 3.80 (d, J=7.8 Hz, 6H, 2×—OCH₃), 3.65 (d, J=4.5 Hz, 6H, 2×—OCH₃), 3.49-3.08 (m, 8H, 4×—CH₂), 2.97-2.58 (m, 4H, 2×—CH₂). MS (ES+) m/z 534 (M+Na)⁺. Anal. calcd. for C₃₂H₃₀FNO₄.

TABLE 1
Biological Activity Data

| Compound | | Molecular | Molecular | EC50 μM | EC50 μM |
|---|---|---|---|---|---|
| No. | STRUCTURE | Formula | Weight | Astrocyte | C6 |
| THI-155 | 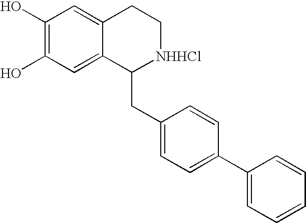 | C₂₂H₂₂ClNO₂ | 367.87 | 7.714 13.23 3.311 9.871 | 3.413 1.286 1.754 0.8821 |
| 4 | 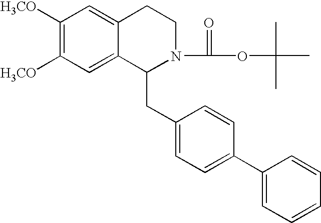 | C₂₉H₃₃NO₄ | 459.58 | 9.811 | 3.896 |
| 5 | 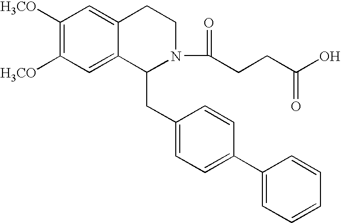 | C₂₈H₂₉NO₅ | 459.53 | No Effect | 10.89 |
| 6 | 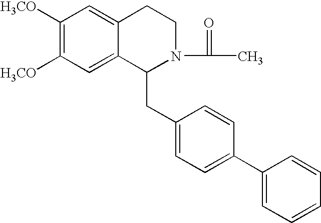 | C₂₆H₂₇NO₃ | 401.50 | 5.226 | 2.480 |
| 7 | 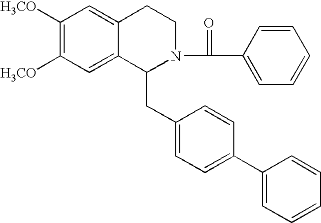 | C₃₁H₂₉NO₃ | 463.57 | 8.713 9.333 | 0.6412 1.561 |
| 8 | 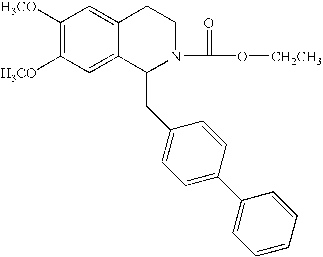 | C₂₇H₂₉NO₄ | 431.52 | 8.253 | 5.985 |
| 9 | 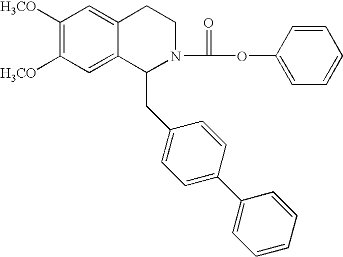 | C₃₁H₂₉NO₄ | 479.57 | 25.65 | 8.504 |
| 10 | 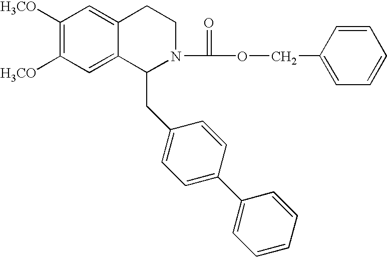 | C₃₂H₃₁NO₄ | 493.59 | 3.638 | 1.108 |
| 11 | 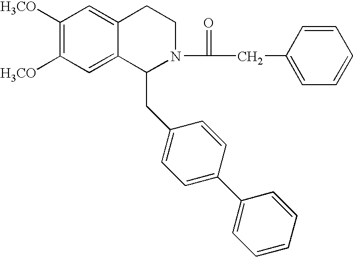 | C₃₂H₃₁NO₃ | 477.59 | 1.349 | 1.242 |
| 14 | 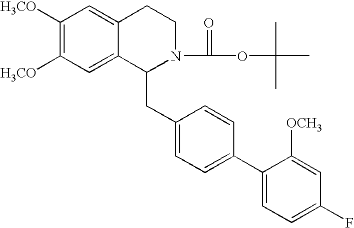 | C₃₀H₃₄FNO₅ | 507.59 | No Effect 2579.000 | 3.055 4.453 |
| 16 | 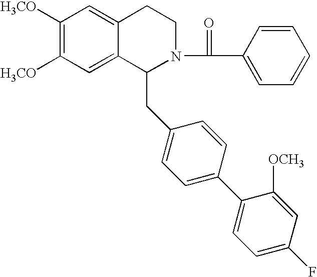 | C₃₂H₃₀FNO₄ | 511.58 | 1.650 4.468 | 0.9156 0.658 |

Structural Modifications to Investigate Pharmacologically Active Sites

Normal cultured rat astrocytes and C6 rat glioma were used as a differential screen for a variety of 1,2,3,4-tetrahydroisoquinoline derivatives. One compound 1-(biphenyl-4-ylmethyl)-1,2,3,4-tetrahydroisoquinoline-6,7-diol (1) selectively blocked the growth of C6 glioma leaving normal astrocytes relatively unaffected. This selectivity against C6 glioma suggested that 1 may have therapeutic potential. When compared to standard chemotherapeutic agents [carmustine (BCNU), 5-fluorouracil, and melphalan], 1 was more potent and comparably selective in differential culture assay. To explore structure activity relationships (SAR) a number of 1,2,3,4-tetrahydroisoquinolines 2, 4, 5, 7, and 8 were synthesized and tested for antiglioma activity. The conformationally flexible analogues 3 (linear tertiary amine) and 6 (linear secondary amine) of the active tetrahydroisoquinolines were also synthesized and evaluated. 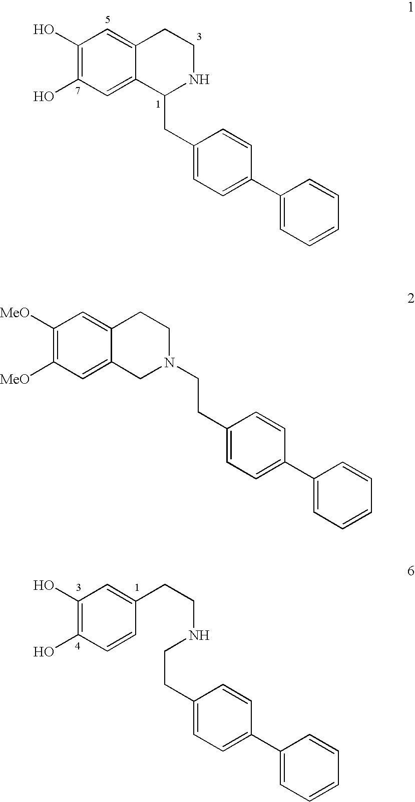
Almost all of the above compounds had selective cytotoxicity against C6 glioma. Of the derivatives described, 1 is the most potent compound, although, as Table 1 indicates, the described compounds are also quite effective. Compound 1 also demonstrates activity against a variety of human glioblastomas. Data obtained to date indicates that 1 may be clinically important for use in the treatment of high-grade gliomas, a form of cancer refractory to current therapeutic agents.

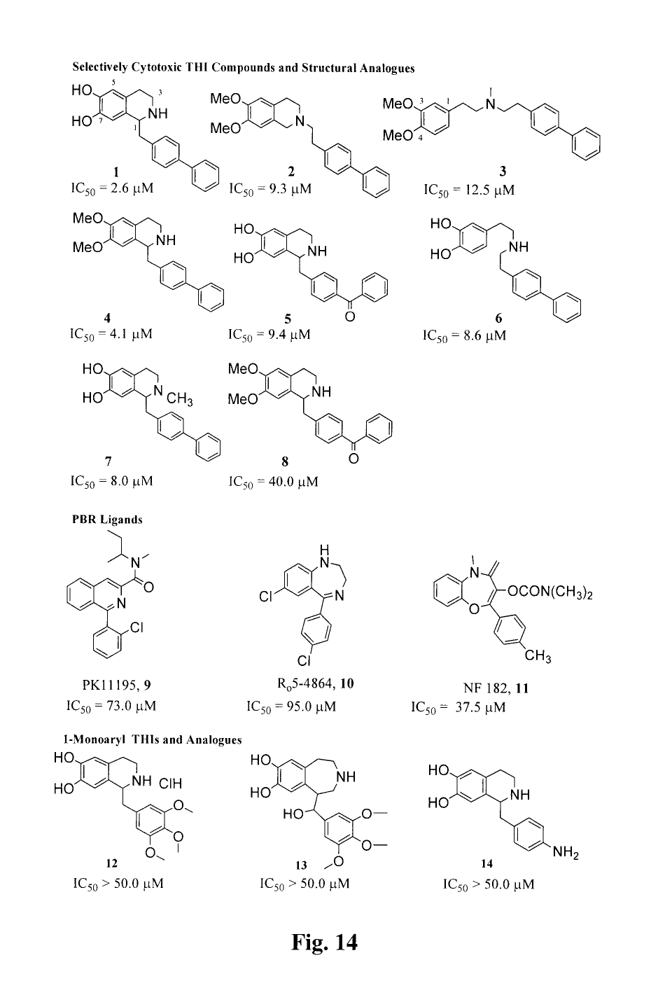

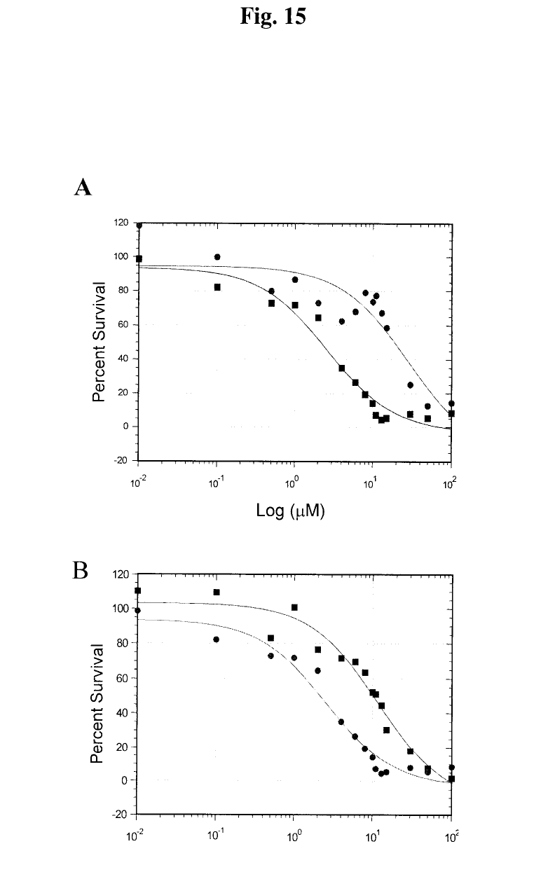

The series of 1,2,3,4-tetrahydroisoquinolines (THI) synthesized by the inventors have selective cytotoxic activity against rat C6 glioma cells relative to cultured rat astrocytes. Compound 1 shown in FIG. 14 was identified in a modified high throughput screening effort comparing the growth of C6 glioma to normal cultured astrocytes. The IC₅₀ values for 1 are approximately 2.6 μM in C6 glioma vs. 29.0 μM in primary astrocytes (see FIG. 15 and Table 2).

TABLE 2
C6 Glioma Selective Cytotoxic Activities of Substituted
Tetrahydroisoquinoline Derivatives

IC₅₀ (μM)^{b,c,d}

| Compound^{a} | C6 Glioma | Astrocytes |
|---|---|---|
| 1 | 2.6 ± 0.6 | 29.0 ± 0.6 |
| | 10.2 ± 1.8 (human) | n/a^{e} |
| 4 | 4.1 ± 0.2 | 83.8 ± 73.2 |
| 5 | 9.4 ± 0.9 | 21.0 ± 7.2 |
| 8 | 40.0 ± 2.6 | 40.0 ± 0.8 |
| 7 | 8.0 ± 0.9 | 15.0 ± 1.3 |
| 2 | 9.3 ± 0.6 | 15.5 ± 1.7 |
| 6 | 8.6 ± 0.5 | >100 |
| 3 | 12.5 ± 0.8 | 39.0 ± 30.7 |
| Melphalan | 12.6 ± 2.6 | 34.0 ± 1.6 |
| BCNU | 9.3 ± 3.9 | 155.6 ± 104.8 |
| 5FU | 6.1 ± 3.9 | >100 |

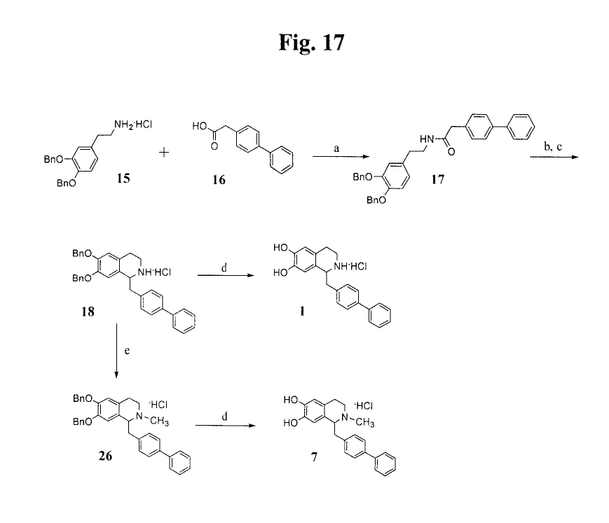

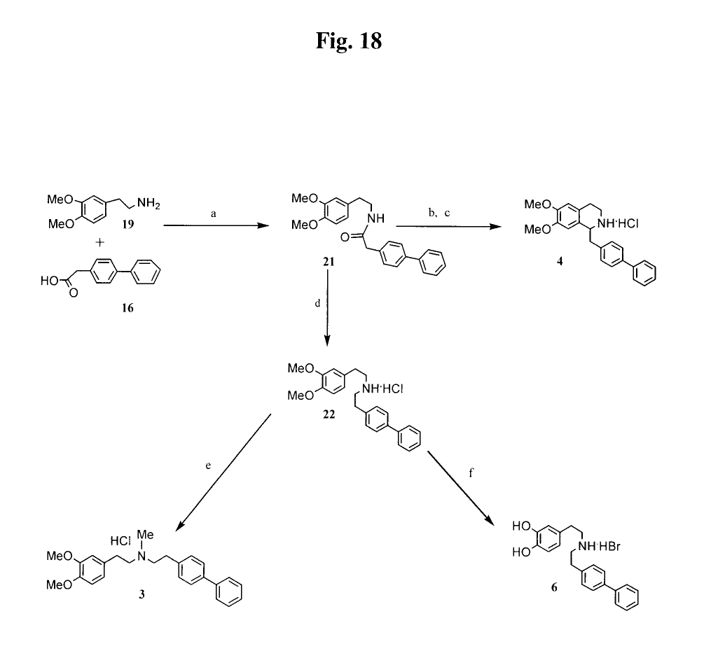

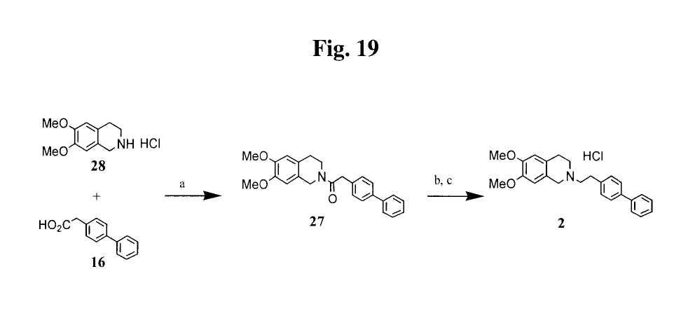

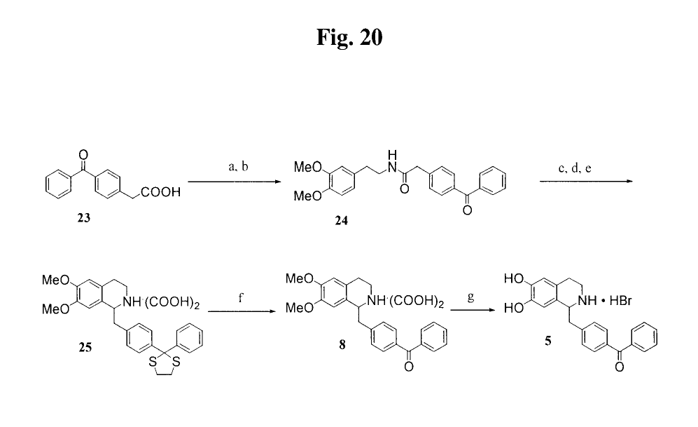

The synthesis of 1-(biphenyl-4-ylmethyl)-1,2,3,4-tetrahydroisoquinoline-6,7-diol 1 is shown in FIG. 17. Reaction of 2-(3,4-debenzyloxyphenyl)ethylamine 15 with 4-biphenylacetic acid 16 in the presence of diethyl cyanosphonate and triethylamine yielded the amide 17. The amide was cyclized to 18 by reaction with POCl₃ followed by reduction with NaBH₄. The lead structure 1 was synthesized by acidic deprotection of 18. The N-methyl analog. 7, was prepared from 18 by treatment with formaldehyde followed by sodium cyanoborohydride and zinc chloride to form 26. Acidic deprotection of 26 to remove the O-benzyl groups protecting the catechol yielded 7. Two types of analogs of 1 were synthesized and tested, substituted THI derivatives such as 2 and open chain derivatives such as 3 (FIG. 14). The synthesis of 1-biphenyl-4-ylmethyl-6,7-dimethoxy-1,2,3,4-tetrahydroisoquinoline 4 is shown in FIG. 18. Initially, the phenethylamine 19 was reacted with 4-biphenylacetic acid 16 to form the intermediate amide 21. The amide 21 was cyclized to form the 6,7-dimethoxy analog 4 by reaction with POCl₃ and NaBH₄ followed by 2 M HCl solution in diethylether. Intermediate 21 was also used to synthesize conformationally flexible analogs 3 and 6. Reaction of 21 with BF₃.Et₂O yielded the linear secondary amine 22. Reaction of 22 with formaldehyde followed by sodium cyanoborohydride and ZnCl₂ yielded the tertiary amine 3. Reaction of 22 with boron tribromide in methylene chloride yielded the secondary amine, catechol 6. The synthesis of 2-(2-biphenyl-4-yl-ethyl)-6,7-dimethoxy-1,2,3,4-tetrahydroisoquinoline 2 is shown in FIG. 19. Reaction of 6,7-dimethoxy-tetrahydroisoquinoline 28 with 4-biphenylacetic acid 16 in the presence of diethyl cyanophosphonate in DMF followed by reaction with triethylamine yielded the amide intermediate 27 (FIG. 19). The amide 27 was reduced using BF₃ Et₂O in THF followed by 1 M B₂H₆ then cold 10% HCl to produce the N-biphenyl tertiary amine, 2 (FIG. 19). The syntheses of 4-(6,7-dimethoxy-1,2,3,4-tetrahydroisoquinoline-1-yl-methyl)-benzophenone oxalate 8 and 4-(6,7-dihydroxy-1,2,3,4-tetrahydroisoquinoline-1-yl-methyl) benzophenone hydrobromide 5 is shown in FIG. 20. The compound 4-benzoylphenylacetic acid 23 was obtained in three steps from p-methylbenzophenone according to the procedure by J. A. Zderic et al. Briefly, acid 23 was transformed into acid chloride by heating with SOCl₂ followed by reaction with 2-(3,4-dimethoxyphenyl) ethylamine in the presence of triethylamine (FIG. 20). The carbonyl group in the resulting amide 24 was protected with 1,2-ethanedithiol using BF₃.Et₂O as a catalyst. Bischler-Napieralski cyclization of the resulting oily product in POCl₃/CH₃CN followed by reduction with NaBH₄ in methanol led to compound 25. The dithiolane ring in compound 25 was smoothly cleaved with mercury(II)perchlorate to form ketone 8. The desired benzophenone 5 was obtained after standard demethylation of compound 8 with BBr₃. All the analogues of 1 synthesized and tested are summarized in FIG. 14.

A high-throughput assay was used to screen libraries of compounds for their effects on the growth of cultured normal astrocytes and C6 glioma. The inventors discovered that C6 glioma growth was more sensitive than normal astrocytes to certain 1,2,3,4-tetrahydroisoquinoline (THI) compounds indicating selective cytotoxic activity in C6 glioma (rat) vs. rat astrocyte. The most active and selective THI screened was 1 (FIG. 14 and FIG. 17). Compound 1 demonstrated at least two-fold higher potency and similar-to-improved selectivity. Next, compound 1 was compared to reported compounds that have similar structure and activity. Two commercially available examples were the PBR ligands 9 and 10. Dose response curves of the prototypic cytotoxic PBR ligands, 9 and 10, and the typical brain cancer chemotherapeutic drugs BCNU, 5-fluorouracil (5FU), and melphalan demonstrated higher IC₅₀ values than 1.

Other tetrahydroisoquinolines (THIs) and analogues were synthesized and tested for activity. Interestingly, a series of 1-position monoaryl substituted tetrahydroisoquinolines and analogues with structures similar to the PBR ligands (FIG. 14) did not show activity in screening assays at 5 μM. In contrast, replacing the biphenyl group at the 1-position of 1 with the benzophenone substituent as in 5 (FIG. 20) resulted in an almost four-fold decrease in activity and decreased selectivity suggesting that C6 cytotoxic activity is relatively intolerant of modification of the biaryl group at the C-1 position.

O-methylation of compound 1 yielded 4 (see FIG. 14), which is two-fold less active than 1. The O-methyl benzophenone analog 8 (FIG. 14) also demonstrated diminished activity and, surprisingly, abolished selectivity compared to 5. The N-methylation of 1 to form 7 resulted in a three-fold decrease in activity and significantly reduced selectivity. Varying the point of attachment of the aromatic moiety also decreased the activity of 2 (FIG. 19) which is two-fold less active than 4.

Conformationally flexible derivatives were synthesized as linear secondary or tertiary amines linking the di-substituted phenyl and biaryl moieties (FIG. 14, compounds 6 and 3). The rationale of synthesizing these compounds was to determine whether these linear analogues could adopt a conformation similar to the THI ring system and therefore bind to the same target site. These open chain analogues demonstrate significant, albeit diminished, anti-glioma activity compared to 1. The linear analog of 1 is 6, which is more than three-fold less active than 1. However it is noteworthy that the selectivity of 6 is preserved or potentially even enhanced. The tertiary amine 3 demonstrated that the cumulative effect of O— and N-methyl groups in the open chain analogues such as 6 was a relatively small decrease in cytotoxicity (≈50%). The N-methyl group may contribute favorably to cytotoxic activity, but in these compounds the cumulative O— and N-methylation demonstrated a detrimental effect on selectivity.

Compound 1 was tested for cytotoxic activity in a human glioma cell line as a proof of principle that cytotoxicity in C6 glioma was indicative of cytotoxicity in human glioma cells. No normal fetal human astrocyte cell lines were readily available, so no negative control was available. Nonetheless, 1 did exhibit activity in the human glioma cell line with an IC₅₀ of 10.2 μM±1.8 μM. Despite the decline in activity relative to C6 glioma (approximately four-fold less active), this indicates that rat C6 glioma activity is indicative of cytotoxicity in human gliomas. Subsequently, compound 1 was demonstrated to have activity in a panel of human gliomas, including U87 and U373.

Melting points were determined on a Thomas-Hoover capillary melting point apparatus and are uncorrected. Infrared spectra were recorded on a Perkin Elmer System 2000 FT-IR. Proton and carbon-13 magnetic resonance spectra were obtained on a Bruker AX 300 spectrometer operating at 300 MHz and 75 MHz for ¹H and ¹³C, respectively. Chemical shift values were reported as parts per million (δ) relative to tetramethylsilane (TMS). Coupling constants (J) are reported in hertz, and the abbreviations for splitting include the following: s, singlet; bs, broad singlet; d, doublet; dd, doublet of doublets; t, triplet; q, quartet; m, multiplet. Spectral data were consistent with assigned structures. Mass spectra were determined on a Bruker-HP Esquire LC System. Elemental analyses were performed by Atlantic Microlab Inc. (Norcross, Ga.), and found values were within ±0.4% of the theoretical values. Routine thin-layer chromatography (TLC) was performed on silica gel on aluminum plates (silica gel 60 F 254, 20×20 cm, Aldrich Chemical Company Inc., Milwaukee, Wis.). Flash chromatography was performed on silica gel (Merck, grade 60, 230-400 mesh, 60 Å). Tetrahydrofuran (THF) was dried by distillation over sodium metal. Acetonitrile (CH₃CN) and methylene chloride (CH₂Cl₂) were dried by distillation from P₂O₅.

### Synthesis of 2-Biphenyl-4-yl-N-[2-(3,4-bis-benzyloxy-phenyl)-ethyl]-acetamide (17)

Triethylamine (2.5 mL, 17.7 mmol) was added dropwise to a stirred solution of 2-(3,4-dibenzyloxyphenyl)ethylamine 15 (2.85 g, 7.7 mmol) and 4-biphenylacetic acid 16 (1.49 g, 7.0 mmol) in 20 mL of anhydrous dimethylformamide at 0° C. Following triethylamine was the dropwise addition of diethyl cyanophosphonate (1.4 mL, 7.7 mmol). The reaction mixture was stirred at room temperature for 22 h and then it was poured into 300 mL of water. The precipitated solid was collected on a glass filter funnel, washed with water (3×70 mL) and air-dried overnight. This solid was recrystallized from hexane/ethanol to give 3.1 g (84%) of 17 as grayish crystals: mp=142-144° C.; ¹H NMR (d₆-DMSO) δ 8.11 (t, J=5.4 Hz, 1H, NH), 7.63-7.59 (m, 2H, ArH), 7.57-7.54 (m, 2H, ArH), 7.46-7.27 (m, 15H, ArH), 6.95-6.92 (m, 2H, ArH), 6.70-6.68 (d, J=8.1 Hz, 1H, ArH), 5.06 (s, 4H, 2×CH₂), 3.41 (s, 2H, CH₂), 3.26 (q, J=5.0 Hz, 2H, CH₂), 2.63 (t, J=2H, CH₂); ¹³C NMR (d₆-DMSO) δ 169.8 (C(O)NH), 148.2, 146.7, 139.9, 138.2, 137.4, 137.3, 135.7, 132.5, 129.4, 128.8, 128.3, 128.2, 127.7, 127.6, 127.5, 127.3, 127.2, 126.5, 126.4, 121.2, 115.2, 114.7, 70.2, 70.1, 42.0, 40.2, 34.5; MS (ES) m/z 550 (M+Na)⁺. Anal. (C₃₆H₃₃NO₃.HCl) C, H, N.

### 6,7-Bis-benzyloxy-1-biphenyl-4-ylmethyl-1,2,3,4-tetrahydroisoquinoline Hydrochloride (18)

A stirred solution of 17 (3 g, 5.69 mmol) and POCl₃ (5.6 mL, 60 mmol) in 25 mL of anhydrous acetonitrile was refluxed for 20 h. The reaction solution was concentrated under reduced pressure and the residue was dissolved in 25 mL of methanol. Sodium borohydride (2.27 g, 60 mmol) was added portionwise to this stirred solution at 0° C. The reaction mixture was stirred at room temperature overnight and then it was poured in 150 mL of 10% aqueous HCl solution. The precipitate was collected on a glass filter funnel, washed with water (3×50 mL) and air-dried overnight. The solid was recrystallized from ether/methanol to give 2.56 g (82%) of 18 as white crystals: mp=187-189° C.; ¹H NMR (d₆-DMSO) δ 9.20 (bs, 1H, NH), 7.73-7.66 (m, 4H, ArH), 7.49-7.21 (m, 15H, ArH), 6.94 (s, 1H, ArH), 6.65 (s, 1H, ArH), 5.13 (s, 2H, PhCH₂), 4.92 (s, 2H, PhCH₂), 4.68-4.62 (m, 1H, CH), 3.37-3.21 (m, 2H, CH₂), 3.00-2.84 (m, 2H, CH₂); ¹³C NMR (d₆-DMSO) δ 147.7, 146.4, 139.7, 138.8, 137.0, 136.9, 135.6, 130.4, 128.9, 128.3, 128.2, 127.7 (2×C), 127.4 (2×C), 127.3, 126.8, 126.5, 124.8, 124.6, 114.2, 112.9, 70.0 (2×C), 54.9, 40.3, 38.5, 24.6; MS (ES) m/z 512 [M+H]⁺. Anal. (C₃₆H₃₃NO₂.HCl) C, H, N.

### 1-(Biphenyl-4-ylmethyl)-1,2,3,4-tetrahydroisoquinoline-6,7-diol hydrochloride (1)

Compound 18 (2.5 g, 4.56 mmol) was stirred in 10 mL of concentrated aqueous HCl solution and 10 mL of methanol. The reaction mixture was refluxed for 10 h and concentrated under reduced pressure and then treated with 10 mL of ether to give a solid. The solid was collected on a glass filter funnel, and washed with ether (2×10 mL) and recrystallized from ether/methanol to give 1.3 g (78%) of 1 as off-white crystals: mp=208-210° C.; ¹H NMR (d₆-DMSO) δ 9.17 (s, 1H, ArOH), 9.09 (s, 1H, NH), 8.90 (s, 1H, ArOH), 7.68 (d, J=7.9 Hz, 4H, ArH), 7.47 (t, J=7.3 Hz, 4H, ArH), 7.36 (t, J=7.3 Hz, 1H, ArH), 6.58 (d, J=4.0 Hz, 2H, ArH), 4.63 (s, 1H, CH), 3.32 (t, J=9.9 Hz, 2H, CH₂), 3.17 (q, J=7.3 Hz, 2H, CH₂), 2.98-2.73 CH₂); 1³C NMR (d₆-DMSO) δ 145.1, 144.0, 139.8, 138.8, 135.4, 130.2, 128.9, 127.4, 126.8, 126.5, 122.7, 122.4, 115.2, 113.5, 55.0, 39.1, 24.2; MS (ES) m/z 332 μM+H]⁺. Anal. (C₂₂H₂₁NO₂ HCl.0.25 H₂O) C, H, N.

### 6,7-Bis-benzyloxy-1-biphenyl-4-ylmethyl-2-methyl-1,2,3,4-tetrahydro-isoquinoline Hydrochloride (26)

Sodium cyanoborohydride (0.11 g, 1.6 mmol) and zinc chloride (0.11 g, 0.8 mmol) in 5 mL of methanol was added to a stirred mixture of 18 (0.81 g, 1.6 mmol) and formaldehyde (5 mmol, 0.4 mL of 37% solution in water) in 7 mL of methanol at room temperature and stirred overnight. The reaction mixture was concentrated under reduced pressure and the residue was mixed with 20 mL of 1 N HCl solution in water. The precipitated solid was collected on a glass filter funnel, washed with water (3×50 mL) and air-dried overnight. It was recrystallized from ether/methanol mixture to give 0.68 g (76%) of 26 as an off-white solid: mp=208-210° C.; ¹H NMR (d₆-DMSO) δ 10.96 (s, 1H, NH⁺), 7.70-7.66 (m, 4H, ArH), 7.46-7.22 (m, 14H, ArH), 7.13-7.06 (m, 2H, ArH), 7.01-6.95 (m, 2H, ArH), 5.96 (s, 1H, ArH), 5.08 (s, 2H, PhCH₂O), 4.64-4.45 (m, 3H, NCH₃), 3.74-3.61 (m, 2H, CH₂), 3.14-2.92 (m, 3H, CH and CH₂), 2.82 (s, 2H, CH₂); ¹³C NMR (d₆-DMSO) δ 147.9, 146.1, 139.5, 138.8, 137.0, 136.6, 135.5, 130.6, 128.9, 128.3, 128.2, 127.8, 127.7, 127.5, 127.4, 127.1, 126.6, 126.5, 122.7, 121.9, 114.0, 113.6, 70.0, 69.8, 63.5, 44.5, 34.3, 24.6, 21.1; MS (ES) m/z 526 [M+H]⁺. Anal. (C₃₇H₃₅NO₂.HCl.0.33H₂O) C, H, N.

### 1-Biphenyl-4-ylmethyl-2-methyl-1,2,3,4-tetrahydroisoquinoline-6,7-diol Hydrochloride (7)

A stirred mixture of 26 (1 g, 1.78 mmol) in 10 mL of concentrated aqueous HCl solution and 10 mL of methanol was refluxed for 10 h. The reaction mixture was concentrated under reduced pressure, the solid residue was mixed with 10 mL of ether, and collected on a glass filter funnel then washed with ether. This solid was recrystallized from ether/methanol to give 0.51 g (75%) of 7 as off-white crystals: mp=232-234° C.; ¹H NMR (d₆-DMSO) δ 10.81 (s, 1H, NH⁺), 9.14 (s, 1H, OH), 8.82 (s, 1H, OH), 7.72-7.62 (m, 4H, ArH), 7.50-7.42 (m, 2H, ArH), 7.39-7.27 (m, 3H, ArH), 6.60 (s, 1H, ArH), 6.09-5.97 (m, 1H, ArH), 4.68-4.50 (m, 1H, CH), 3.67-3.21 (m, 2H, CH₂), 3.08-2.74 (m, 7H, 2×CH₂ and CH₃); ¹³C NMR (d₆-DMSO) δ 145.4, 143.8, 139.7, 139.6, 138.6, 135.5, 130.2, 128.9, 128.8 (2×C), 127.4, 126.6, 126.5, 126.4, 120.4, 120.0, 115.3, 114.8, 63.5, 44.5, 20.7; MS (ES) m/z 346 [M+H]⁺. Anal. (C₂₃H₂₃NO₂.HCl.0.33H₂O) C, H, N.

### 2-Biphenyl-4-yl-N-[2-(3,4-dimethoxy-phenyl)-ethyl]-acetamide (21)

Diethyl cyanophosphonate (1.99 g, 11 mmol) followed by triethylamine (1.7 mL, 12 mmol) was added dropwise to a stirred solution of 2-(3,4-dimethoxyphenyl)-ethylamine 19 (1.81 g, 10 mmol) and of 4-biphenylacetic acid 16 (1.91 g, 9 mmol) in 40 mL of DMF at room temperature. The reaction mixture was stirred at room temperature overnight and poured into 1 L of water. The precipitated solid was collected on a glass filter funnel, washed with water (3×50 mL) and air-dried overnight. It was recrystallized from hexane/ethyl acetate to yield 2.77 g (82%) of 21 as a white solid, mp=103-105° C. (lit. mp=° C.).

### 1-Biphenyl-4-ylmethyl-6,7-dimethoxy-1,2,3,4-tetrahydroisoquinoline Hydrochloride (4)

POCl₃ (7.67 g, 50 mmol) was added to a stirred solution of amide 21 (1.88 g, 5 mmol) in 20 mL of anhydrous acetonitrile and refluxed for 22 h. The reaction solution was concentrated under reduced pressure and the residue was dissolved in 20 mL of methanol. Sodium borohydride (1.89 g, 50 mmol) was added portionwise to this stirred solution at 0° C. The reaction mixture was stirred overnight at room temperature and then poured into 100 mL of 10% aqueous HCl solution. The resultant mixture was washed with 150 mL of chloroform. The organic layer was washed with 80 mL of water, 80 mL of brine, and dried (Na₂SO₄). The solvent was concentrated under reduced pressure and the residue was mixed with 50 mL of 2 M HCl solution in anhydrous ether. The precipitate formed was collected on a glass filter funnel, washed with ether (3×50 mL) and air-dried overnight. The solid was recrystallized from ether/methanol to give 1.64 g (83%) of 4 as white crystals, mp=235-237° C. (lit. mp=236-237° C.).

### 2-Biphenyl-4-yl-ethyl-[2-(3,4-dimethoxyphenyl)-ethyl]-amine Hydrochloride (22)

Boron trifluoride diethyl etherate (1.3 mL, 10 mmol), followed by 25 mL of 1 M B₂H₆ solution in THF was added to a stirred solution of amide 21 (1.88 g, 5.0 mmol) in 35 mL of anhydrous THF at room temperature. The reaction solution was refluxed overnight. The solvent was evaporated under reduced pressure and the residue was mixed with cold 1 M aqueous HCl solution. The precipitated solid was collected on a glass filter funnel, washed with water and air-dried overnight. It was recrystallized from ether/methanol to give 1.41 g (71%) of 22 as a white solid, mp=222-224° C.; ¹H NMR (d₆-DMSO) δ 9.25 (s, 2H, NH), 7.69-7.59 (m, 4H, ArH), 7.46 (t, J=7.4 Hz, 2H, ArH), 7.36 (d, J=7.4 Hz, 3H, ArH), 6.90 (d, J=8.3 Hz, 1H, ArH), 6.88 (s, 1H, ArH), 6.77 (d, J=8.1 Hz, 1H, ArH), 3.75 (s, 3H, OCH₃), 3.72 (s, 3H, OCH₃), 3.23-3.11 (m, 4H, CH₂), 3.08-3.02 (m, 2H, CH₂), 2.97-2.88 (m, 2H, CH₂); ¹³C NMR (d₆-DMSO) δ 148.8, 147.7, 139.8, 138.6, 136.5, 129.6, 129.2, 128.9, 127.3, 126.8, 126.5, 120.5, 112.5, 112.1, 55.5, 55.4, 47.9, 47.6, 31.1, 31.0; MS (ES) m/z 362 [M+H]⁺. Anal. (C₂₄H₂₆NO₂.HCl) C, H, N.

### 2-Biphenyl-4-yl-ethyl-[2-(3,4-dimethoxyphenyl)-ethyl]-methylamine hydrochlor-ide (3)

A solution of sodium cyanoborohydride (0.08 g, 1.3 mmol) and zinc chloride (0.09 g, 0.65 mmol) in 4 mL of methanol was added to a stirred mixture of 22 (0.47 g, 1.3 mmol) and formaldehyde (4 mmol, 0.35 mL of 37% solution in water) in 5 mL of methanol at room temperature. The reaction mixture was stirred overnight at room temperature. The solvent was evaporated under reduced pressure and the residue was mixed with 15 mL of 1 N HCl solution in water. The precipitated solid was collected on a glass filter funnel, washed with water, and air-dried overnight. It was recrystallized from ether/methanol to give 0.38 g (72%) of 3 as an off-white solid, mp=183-185° C.; ¹H NMR (d₆-DMSO) □ 10.79 (s, 1H, NH), 7.68-7.61 (m, 4H, ArH), 7.48-7.33 (m, 5H, ArH), 6.96-6.92 (m, 2H, ArH), 6.80 (d, J=10.0 Hz, 1H, ArH), 3.75 (s, 3H, OCH₃), 3.72 (s, 3H, OCH₃), 3.43-3.20 (m, 4H, 2×CH₂′), 3.11 (t, J=8.4 Hz, 2H, CH₂), 3.00 (t, J=8.4 Hz, 2H, CH₂), 2.88 (d, J=3.9 Hz, 3H, NCH₃); ¹³C NMR (d₆-DMSO) δ 148.8, 147.7, 139.7, 138.7, 136.3, 129.4, 129.3, 128.9, 127.3, 126.8, 120.7, 112.7, 112.1, 56.0, 55.7, 55.5, 39.1, 29.0, 28.0; MS (ES) m/z 376 [M+H]⁺. Anal. (C₂₅H₂₉NO₂.HCl) C, H, N.

### 4-[2-(2-Biphenyl-4-yl-ethylamino)-ethyl]-benzene-1,2-diol hydrobromide (6)

A 1 M boron tribromide solution (3.2 mL, 3 mol. equiv.) was added with stirring to a solution of 22 (0.57 g, 1.57 mmol) in 15 mL of anhydrous methylene chloride under argon at −70° C. The mixture was stirred overnight at room temperature and the solvent was evaporated under reduced pressure. The residue was dissolved in 15 mL of methanol and, again, the solvent was evaporated under reduced pressure. The solid residue was mixed with 10 mL of ether, collected on a glass filter funnel, washed with ether (3×20 mL) to give a solid, which was recrystallized from ether/methanol to give 0.49 g (75%) of 6 as an off-white solid, mp=203-205° C.; ¹H NMR (d₆-DMSO) δ 8.85 (s, 1H, ArOH), 8.83 (s, 1H, ArOH), 8.58 (bs, 2H, NH), 7.68-7.62 (m, 4H, ArH), 7.46 (t, J=7.5 Hz, 2H, ArH), 7.39-7.34 (m, 3H, ArH), 6.68 (d, J=8.0 Hz, 1H, ArH), 6.63 (d, J=2.0 Hz, 1H, ArH), 6.49 (d, J=8.0 Hz, 1H, ArH), 3.21 (s, 2H, CH₂), 3.11 (s, 2H, CH₂), 2.96 (t, J=8.1 Hz, 2H, CH₂), 2.75 (t, J=8.0 Hz, 2H, CH₂); ¹³C NMR (d₆-DMSO) δ 145.3, 144.1, 139.8, 138.7, 136.3, 129.2, 128.9, 127.4, 126.9, 126.5, 119.2, 116.0, 115.7, 48.2, 47.6, 31.2, 31.0; MS (ES) m/z 334 [M+H]⁺. Anal. (C₂₂H₂₃NO₂.HBr) C, H, N.

### 2-Biphenyl-4-yl-1-(6,7-dimethoxy-3,4-dihydro-1H-isoquinolin-2-yl)-ethanone (27)

Diethyl cyanophosphonate (1.63 g, 9 mmol) followed by triethylamine (2.8 mL, 20 mmol) was added dropwise to a stirred mixture of 6,7-dimethoxy-1,2,3,4-tetrahydroisoquinoline hydrochloride 28 (1.94 g, 8.5 mmol) and 4-biphenyl acetic acid 16 (1.5 g, 7 mmol) in 50 mL of DMF at room temperature. The reaction mixture was stirred at room temperature overnight and poured into 1 L of water. The precipitated solid was collected on a glass filter funnel, washed with water and air-dried overnight. It was recrystallized from hexane/ethyl acetate to give 2.17 g (80%) of 27 as a white solid: mp=109-111° C.; ¹H NMR (d₆-DMSO) δ 7.68-7.56 (m, 4H, ArH), 7.45 (t, J=7.6 Hz, 2H, ArH), 7.36-7.32 (m, 3H, ArH), 6.78-6.71 (m, 2H, ArH), 4.65 (s, 1H, PhCH₂CO), 4.55 (s, 1H, PhCH₂CO), 3.83 (s, 2H, PhCH₂N), 3.72-3.65 (m, 8H, 2×OCH₃ and PhCH₂), 2.67 (q, J=6.0 Hz, 2H, CH₂N); ¹³C NMR (d₆-DMSO) δ 169.1 (CONH), 147.5, 147.4, 147.3, 147.2, 139.9, 138.2, 135.2, 129.7, 129.6, 128.8, 127.2, 126.5, 126.0, 125.2, 124. 9, 112.0, 111.8, 110.1, 55.5, 46.6, 43.4, 43.2, 28.2, 27.4; MS (ES) m/z 410 [M+Na]⁺. Anal. (C₂₅H₂₅NO₃) C, H, N.

### 2-(2-Biphenyl-4-yl-ethyl)-6,7-dimethoxy-1,2,3,4-tetrahydroisoquinoline (2)

Boron trifluoride diethyl etherate (1.2 mL, 9.47 mmol) followed by 20 mL of 1 M B₂H₆ solution in THF was added to a stirred solution of 27 (1.8 g, 4.65 mmol) in 30 mL of anhydrous THF at room temperature and refluxed overnight. The solvent was concentrated under reduced pressure and the residue was treated with cold 1% aqueous HCl solution. The precipitated solid was collected on a glass filter funnel, washed with water, and air-dried overnight. It was recrystallized from methanol/acetic acid to give 1.22 g (70%) of 2 as a white solid: mp=162-164° C.; ¹H NMR (d₆-DMSO) δ 7.62-7.56 (m, 4H, ArH), 7.46-7.41 (m, 2H, ArH), 7.36-7.26 (m, 3H, ArH), 6.76 (s, 1H, ArH), 6.70 (s, 1H, ArH), 3.97 (s, 2H, PhCH₂N), 3.72 (s, 3H, OCH₃), 3.69 (s, 3H, OCH₃), 3.21-3.10 (m, 4H, CH₂), 2.92-2.87 (m, 4H, CH₂); ¹³C NMR (d₆-DMSO) δ 147.8, 147.4, 139.8, 138.3, 137.9, 129.2, 128.8, 127.2, 126.8, 126.5, 123.3, 122,2, 111.5, 110.2, 59.8, 59.5, 55.5, 55.4, 54.2, 29.3, 23.5; MS (ES) m/z 374 [M+H]⁺. Anal. (C₂₅H₂₇NO₂.6H₂O) C, H, N.

### N-(3,4-Dimethoxyphenethyl)-4-benzoylphenylacetamide (24)

A solution of p-benzoylphenylacetic acid 23 (4.81 g, 0.02 mol) and SOCl₂ (2.46 mL, 0.034 mol) in 40 mL of benzene was heated to reflux for 3 h. The reaction mixture was concentrated 2 times with benzene, dissolved in 50 mL of CH₂Cl₂, cooled on an ice bath and 2-(3,4-dimethoxyphenyl)-ethylamine 19 (3.99 g, 0.02 mol) was added dropwise. Triethylamine (4.05 g, 0.04 mol) was added and the reaction mixture was stirred overnight at room temperature, washed with 1 N HCl, H₂O, 1 N NaOH, and H₂O, dried over MgSO₄, and evaporated. The resulting oil was recrystallized from CH₂Cl₂/hexane to give 6.72 g (83%) of 24 as a white solid: mp=113-114° C.; ¹H NMR (d₆-DMSO) δ 7.81-7.72 (m, 4H, ArH), 7.67-7.55 (m, 1H, ArH), 7.53-7.46 (m, 2H, ArH), 7.32 (d, J=8.3 Hz, 2H, ArH), 6.73 (d, J=8.1 Hz, 1H, ArH), 6.65 (d, J=1.9 Hz, 1H, ArH), 6.56 (dd, J=8.1 Hz, 1.9 Hz, 1H, ArH), 5.55-5.43 (m, 1H, NH), 3.83 (s, 3H, MeO), 3.82 (s, 3H, MeO), 3.60 (s, 2H, CH₂CO), 3.52-3.47 (m, 2H, CH₂N), 2.71 (t, J=6.9 Hz, 2H, CH₂Ar); 1³C NMR (d₆-DMSO) δ 196.1 (C═O), 169.9 (NCO), 149.1, 147.8, 139.6, 137.4, 136.6, 132.5, 130.9, 130.6, 129.9, 129.3, 128.3, 120.6, 111.8, 111.3, 55.9, 55.8, 43.7, 40.8, 35.0; IR (KBr) 3327 (NH), 1659 (C═O), 1642 (C═O, amide), 1549, 1518 cm⁻¹; MS (ES) m/z 426 [M+Na]⁺. Anal. (C₂₅H₂₅NO₄.0.05 CH₂Cl₂) C, H, N.

### 6,7-Dimethoxy-1-[4-(2-phenyl-1,3-dithiolane-2-yl)benzyl]-1,2,3,4-tetrahydro-isoquinoline Oxalate (25)

A solution of the amide 24 (4.84 g, 12 mmol), 1,2-ethanedithiol (7.2 mL, 82 mmol), and boron trifluoride diethyl etherate (7.2 mL, 59 mmol) in 18 mL of anhydrous CHCl₃ was stirred overnight at room temperature under argon atmosphere. CHCl₃ was added, washed 3 times with saturated Na₂CO₃, brine, dried over MgSO₄, and concentrated. The resulting oil was dissolved in 50 mL of CH₃CN and reconcentrated, CH₃CN (85 mL) and POCl₃ (7.8 mL) were added and refluxed for 5 h. The solution was concentrated and evaporated with CH₃OH (3×50 mL). The oil was dissolved in 125 mL of CH₃OH and NaBH₄ (9.6 g, 0.25 mol) was added in small portions. The reaction mixture was stirred overnight at room temperature. CH₃OH was evaporated, CHCl₃ (150 mL) added and washed twice with 10% NaOH (100 mL), H₂O (100 mL), dried over MgSO₄, filtered, and concentrated. The product was dissolved in CH₂Cl₂ (20 mL) and MeOH (20 mL) and solution of oxalic acid (1.8 g, 14 mmol) in minimal amount of MeOH (10 mL) was added followed by ether (100 mL). The product was subsequently filtered and washed with ether to give 3.93 g (59%) of 25 as white crystals: mp=151-152.5° C.; ¹H NMR (d₆-DMSO) δ 7.54-7.43 (m, 4H, ArH), 7.36-7.20 (m, 5H, ArH), 6.77 (s, 1H, ArH), 6.17 (s, 1H, ArH), 4.68-4.59 (m, 1H, CH), 3.71 (s, 3H, CH₃O), 3.50-3.39 (m, 1H, CH), 3.40 (s, 4H, 2×SCH₂), 3.39 (s, 314, CH₃O), 3.31-3.12 (m, 3H, CH₂ and CH), 2.98-2.85 (m, 2H, CH); ¹³C NMR (d₄-CH₃OH) δ 150.5, 149.0, 146.2, 145.8, 136.1, 130.5, 129.9, 129.3, 128.9, 128.3, 124.9, 124.6, 113.0, 111.5, 57.2, 56.4, 41.1, 41.0, 40.8, 39.9, 25.8; IR (KBr) 3300-2300 (br, NH+OH), 1721 (C═O, acid), 1614, 1520 cm−1; MS (ES) m/z 464 [M+H]⁺. Anal. (C₂₇H₂₉NO₂S₂.(COOH)₂) C, H, N.

### 4-(6,7-Dimethoxy-1, 2,3,4-tetrahydroisoquinoline-1-yl-methyl)benzophenone Oxalate (8)

The tetrahydroisoquinoline oxalate salt 25 (3.81 g, 6.88 mmol) was mixed with 100 mL of 1 N NaOH, extracted with 150 mL of CHCl₃, washed with 100 mL of brine, dried (MgSO₄), filtered, and concentrated. The resulting oil was dissolved in 75 mL of CHCl₃ and a solution of Hg(ClO₄)₂.×H₂O (9.61 g, about 21 mmol) in MeOH (75 mL) was added and stirred for 1 h. A yellow precipitate was removed by filtering through celite. The filtrate was extracted with 200 mL of CHCl₃, washed with brine (100 mL), sat. Na₂CO₃ (100 mL), brine (100 mL), dried over MgSO₄, filtered, concentrated to an approximate 10 mL volume. A solution of oxalic acid (1.26 g, 10 mmol) in MeOH (10 mL) was added followed by ether (100 mL). After cooling, the product was filtered and washed with ether to give 2.35 g (72%) of 8 as white crystals: mp=201-202° C.; ¹H NMR (d₆-DMSO) δ 8.24 (s, NH), 7.76-7.65 (m, 5H, ArH), 7.61-7.49 (m, 4H, ArH), 6.79 (s, 1H, ArH), 6.49 (s, 1H, ArH), 4.73 (t, J=7.0 Hz, 1H, CH), 3.73 (s, 3H, MeO), 3.54 (s, 3H, MeO), 3.49-3.38 (m, 2H, CH), 3.32-3.19 (m, 2H, CH), 3.01-2.82 (m, 2H, CH); ¹³C NMR (d₆-DMSO) □195.5 (C═O), 148.2, 147.0, 141.6, 137.1, 135.8, 132.7, 130.01, 129.9, 129.5, 128.6, 124.2, 124.0, 111.7, 110.1, 55.4, 55.3, 54.8, 38.4, 24.7; IR (KBr) 3300-2300 (NH, OH), 1721 (C═O, acid), 1657 (C═O), 1609, 1521 cm⁻; MS (ES) m/z 388 [M+H]⁺. Anal. (C₂₅H₂₅NO₃.(COOH)₂) C, H, N.

### 4-(6,7-Dihydroxy-1,2,3,4-tetrahydroisoquinoline-1-yl-metllyl)benzophenone Hydrobromide (5)

The free base was obtained by extraction of a solution of tetrahydroisoquinoline oxalate salt 8 (0.478 g, 1 mmol) in 1 N NaOH with CHCl₃, dried with MgSO₄, evaporated, and dried in vacuum. The resulting oil was dissolved in anhydrous CH₂Cl₂ (20 mL) under an argon atmosphere and cooled down with dry ice-acetone bath. Solution of 1 N BBr₃ (4.0 mL, 4 mmol) in CH₂Cl₂ was added with syringe and stirred overnight at room temperature. The solution was cooled down again followed by addition of 10 mL of MeOH and stirred for 4 h at room temperature, then evaporated 5 times with MeOH almost to dryness. MeOH/ether was added to crystals and the refrigerated crystals were collected by filtration and washed with MeOH/ether to give 0.39 g (88%) of 5 as white crystals: mp=239-241° C.; ¹H NMR (d₄-CH₃OH) δ 7.91-7.78 (m, 4H, ArH), 7.65 (s, 1H, ArH), 7.58-7.48 (m, 4H, ArH), 6.64 (s, 1H, ArH), 6.52 (s, 1H, ArH), 3.60-3.50 (m, 3H, CH₂ and CH), 3.37-3.19 (m, 2H, CH₂), 2.88-2.32 (m, 2H, CH₂); ¹³C NMR (d₄-CH₃OH) δ 198.2 (C═O), 147.1, 145.8, 142.0, 138.7, 138.2, 133.9, 131.8, 131.0, 130.9, 129.6, 123.7, 123.3, 116.3, 114.4, 55.5, 41.1, 40.9, 25.6; IR (KBr) 3600-2600 (OH+NH), 1646 (C═O), 1608 (C═C, Ar), 1527, 1282 cm⁻¹; MS (ES) m/z 360 [M+H]⁺. Anal. (C₂₃H₂₁NO₃.HBr) C, H, N.

The compounds tested were synthesized in the laboratory of Dr. Duane D. Miller, The University of Tennessee, Memphis, Tenn. Reference compounds were purchased from Sigma-Aldrich and include carmustine[1,2-bis(2-chloroethyl)-1-nitrosourea, BCNU], 5-fluorouracil (5FU), melphalan (3-(p-(bis(2-chloroethyl)amino)phenyl)-L-alanine), 9 (1-(2-chlorophenyl(-N-methyl-N-(1-methyl-propyl)-3-isoquinoline carboxamide, PK11195), and 10 (7-chloro-5-(4-chlorophenyl)-2,3-dihydro-1H-benzo[e][1,4]diazepine, R_{O}5-4864).

The astrocytes primary culture was derived from neonatal (4-8 days postpartum) rat pups as described by Geisert et al. The primary cultures were prepared as per McCarthy et al. Briefly, the rat pups were anesthetized on ice, decapitated, and the brains were removed. The cerebral cortex was removed, stripped of the meninges, and cut into small pieces of approximately 1 mm². The tissue was placed in 0.1% trypsin for 15 min and gently pipetted to produce a single cell suspension. The cell suspension was then rinsed in media and transferred to culture flasks. The cells were maintained in 10% fetal calf serum (FCS; Hyclone®) Basal Medium Eagle (BME, Gibco®). The cells were cultured in 75 cm² flasks, passaged to a new flask, and grown for several weeks before use. C6 rat glioma and T98G human glioma cells were derived from frozen cultures of cells bought from ATCC.

Screening assays were performed as per Wagner, et al. Briefly, the primary cultures of astrocytes and clonal cultures of C6 glioma were handled identically with respect to treatment concentrations and manipulations of cells for the screening assays. The cells were trypsinized and transferred to 96-well microtiter plates at an approximate cell density of 10³ cells/mm² in the wells. The effect of cell plating density on cell growth was investigated by doing identical experiments at different cell densities. The cell density range used in screening assays and dose response experiments does not cause variability in the growth rate of C6 or primary astrocyte cultures. The cells were grown overnight in 100 μL of 10% FCS BME in a 37° C. incubator containing a humid, 5% CO₂ atmosphere.

All samples were dissolved completely to make a 100 μM stock solution and diluted to produce a series of concentrations. In some cases, solubility problems were addressed by dissolving compounds in ethanol or DMSO (≦1% final concentration) then diluting them into the assay media. Immediately before treatment, the 10% FCS BME was suctioned off the cells and replaced with the 190 μL of 2% FCS BME. Ten tiL aliquots of the diluted stock solutions were added to the 190 μL in the wells to produce the test concentration. The vehicle solution was tested as a control and dilutions were performed such that co-solvent concentrations did not varying between wells.

The experimental treatment was the incubation of the cells with test compound for 96 h at 37° C., 5% CO₂. The cells were fixed with 4% formaldehyde, stained with 0.1% cresylecth violet stain, and quantitated as per Wagner et a. The screening data was collected as four wells per compound (screening assays) or concentration (dose response curves). Also, the average growth of 8 wells with no treatment was used as a negative control for each plate.

The cytotoxic character of each compound was reported as the percent survival, calculated as the average A₅₆₀ for treated cells divided by A₅₆₀ of untreated (negative control) cells, and expressed as a percentage. Values less than 100% indicate some degree of cytotoxicity. Dose response curves and IC₅₀ values were attained via plots of percent survival vs. concentration. The raw data was input for an inhibitory activity model in the program WinNonlin® (Pharsight© Corporation). The curve drawn for each experiment represents the best-fit curve derived from WinNonlin. Up to sixteen concentrations per test compound were used as input for the calculation of the IC₅₀ values. Promising compounds in this assay would demonstrate selective cytotoxicity in the C6 glioma cell lines at levels lower than current chemotherapeutic and other glioma toxic agents. Compound 1 and BCNU were characterized further in the dose response curves in FIG. 15 that were generated using Scientist® (MicroMath® Inc.) software Version 2.01.

Preferred compositions and methods for their synthesis have been depicted and described in detail herein, but it should be apparent to those skilled in the relevant art that various modifications, additions, substitutions, and the like can be made without departing from the invention or falling outside its scope.

What is claimed is:

1. A compound according to formula (I) 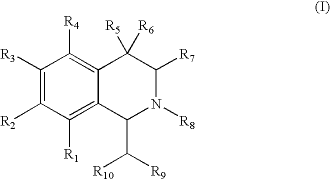
wherein,
R₁, R₂, R₃, and R₄ are independently hydrogen or hydroxyl;
R₅, R₆, and R₇ are independently hydrogen or alkyl;
R₈ is hydrogen, alkyl, aryl, arylalkyl, 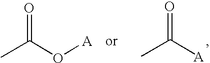
where A is substituted or unsubstituted alkyl, aryl, alkenyl, alkoxy, alkyloxy, or arylalkyl. 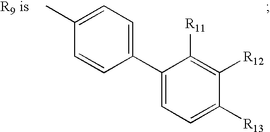
R₁₀ is hydrogen; and
R₁₁ and R₁₂ and R₁₃ are independently hydrogen, hydroxyl, halide, alkyl, arylalkyl, alkenyl, arylalkenyl, alkoxy, aryloxy, amino, alkylamino, dialkylamino, arylamino, aryl, or cyclohexyl.

2. A compound as in claim 1 wherein when 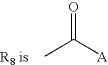
is aryl.

3. A compound as in claim 2 wherein when R₈ is,

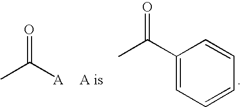

4. A compound as in claim 1 wherein when R₈ is 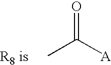
is alklyl.

5. A compound as in claim 1 wherein when 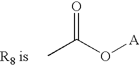
is alkyl.

6. A method for treating cancer, the method comprising administering to a patient a compound according to formula (I) 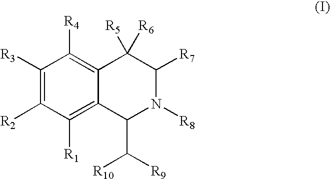
wherein,
R₁, R₂, R₃, and R₄ are independently hydrogen or hydroxyl;
R₅, R₆, and R₇ are independently hydrogen or alkyl;
R₈ is hydrogen, alkyl, aryl, arylalkyl, 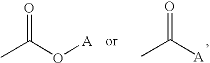
where A is substituted or unsubstituted alkyl, aryl, alkenyl, alkoxy, alkyloxy, or arylalkyl. 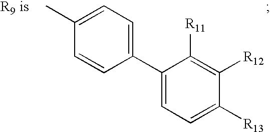
R₁₀ is hydrogen; and
R₁₁ and R₁₂ and R₁₃ are independently hydrogen, hydroxyl, halide, alkyl, arylalkyl, alkenyl, arylalkenyl, alkoxy, aryloxy, amino, alkylamino, dialkylamino, arylamino, aryl, or cyclohexyl.

7. The method of claim 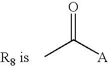
A is aryl.

8. A compound as in claim 2 wherein when 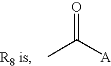
A is

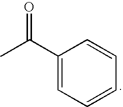

9. A compound as in claim 1 wherein when 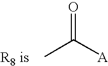
is alklyl.

10. A compound as in claim 1 wherein when 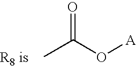
A is alkyl.
